SEQUENCE LISTING (18 sequences)

SEQ ID NO: 1 - protein, 31 aa, synthetic construct
  MOD_RES 2: note = Aib(Aminoisobutyric acid)
  MOD_RES 13: note = Aib(Aminoisobutyric acid)
YXEGTFISDYSIXLDKIHQEDFVKWLLAQKG

SEQ ID NO: 2 - protein, 31 aa, synthetic construct
  MOD_RES 2: note = Aib(Aminoisobutyric acid)
  MOD_RES 13: note = Aib(Aminoisobutyric acid)
YXEGTFISDYSIXLDEIHQKDFVKWLLAQKG

SEQ ID NO: 3 - protein, 31 aa, synthetic construct
  MOD_RES 2: note = Aib(Aminoisobutyric acid)
YXEGTFISDYSIALDKIHQEDFVKWLLAQKG

SEQ ID NO: 4 - protein, 31 aa, synthetic construct
  MOD_RES 2: note = Aib(Aminoisobutyric acid)
YXEGTFISDYSIALDEIHQKDFVKWLLAQKG

SEQ ID NO: 5 - protein, 31 aa, synthetic construct
  MOD_RES 2: note = Aib(Aminoisobutyric acid)
YXEGTFISDYSIALDEIHQKDFVNWLLAQRG

SEQ ID NO: 6 - protein, 31 aa, synthetic construct
  MOD_RES 2: note = Aib(Aminoisobutyric acid)
YXEGTFISDYSIALDELHQKDFVNWLLAQRG

SEQ ID NO: 7 - protein, 31 aa, synthetic construct
  MOD_RES 2: note = Aib(Aminoisobutyric acid)
YXEGTFISDYSIALDKIHQQDFVNWLLAQRG

SEQ ID NO: 8 - protein, 31 aa, synthetic construct
  MOD_RES 2: note = Aib(Aminoisobutyric acid)
YXEGTFISDYSIALDKLHQQDFVNWLLAQRG

SEQ ID NO: 9 - protein, 31 aa, synthetic construct
  MOD_RES 2: note = Aib(Aminoisobutyric acid)
YXEGTFISDYSIALDKIHQEDFVNWLLAQRG

SEQ ID NO: 10 - protein, 31 aa, synthetic construct
  MOD_RES 2: note = Aib(Aminoisobutyric acid)
YXEGTFISDYSIALDKLHQEDFVNWLLAQRG

SEQ ID NO: 11 - protein, 31 aa, synthetic construct
  MOD_RES 2: note = Aib(Aminoisobutyric acid)
YXEGTFISDYSIALDRIHQEDFVKWLLAQRG

SEQ ID NO: 12 - protein, 31 aa, synthetic construct
  MOD_RES 2: note = Aib(Aminoisobutyric acid)
YXEGTFISDYSIALDRIHQEDFVNWLLAQKG

SEQ ID NO: 13 - protein, 31 aa, synthetic construct
  MOD_RES 2: note = Aib(Aminoisobutyric acid)
YXEGTFISDYSIALDRIHQQDFVKWLLAQRG

SEQ ID NO: 14 - protein, 31 aa, synthetic construct
  MOD_RES 2: note = Aib(Aminoisobutyric acid)
YXEGTFISDYSIALDRIHQQDFVNWLLAQKG

SEQ ID NO: 15 - protein, 31 aa, synthetic construct
  MOD_RES 2: note = Aib(Aminoisobutyric acid)
YXEGTFISDYSIALDRLHQEDFVKWLLAQRG

SEQ ID NO: 16 - protein, 31 aa, synthetic construct
  MOD_RES 2: note = Aib(Aminoisobutyric acid)
YXEGTFISDYSIALDRLHQEDFVNWLLAQKG

SEQ ID NO: 17 - protein, 31 aa, synthetic construct
  MOD_RES 2: note = Aib(Aminoisobutyric acid)
YXEGTFISDYSIALDRLHQQDFVKWLLAQRG

SEQ ID NO: 18 - protein, 31 aa, synthetic construct
  MOD_RES 2: note = Aib(Aminoisobutyric acid)
YXEGTFISDYSIALDRLHQQDFVNWLLAQKG

(19) United States
(12) Patent Application Publication
(10) Pub. No.: US 2024/0239863 A1
(43) Pub. Date: Jul. 18, 2024

(54) GIP RECEPTOR AGONIST AND USE THEREOF

(71) Applicant: HANGZHOU ZHONGMEIHUADONG PHARMACEUTICAL CO., LTD., Zhejiang (CN)

(72) Inventors: Honghao LI, Zhejiang (CN); Wenchen LI, Zhejiang (CN); Haisong XIE, Zhejiang (CN); Yingchi ZHAO, Zhejiang (CN); Hongmei XU, Zhejiang (CN)

(21) Appl. No.: 18/586,899

(22) Filed: Feb. 26, 2024

(30) Foreign Application Priority Data
Dec. 26, 2022 (CN) 202211673990.6
Dec. 21, 2023 (CN) 202311769474.8

(51) Int. Cl. C07K 14/645; A61K 9/00; A61K 38/00; A61K 47/54; A61P 3/10

(52) U.S. Cl. CPC C07K 14/645; A61K 9/0019; A61K 47/542; A61P 3/10; A61K 38/00

(57) ABSTRACT

The present invention is in the field of biomedical technology, and discloses a peptide compound having agonistic activity at glucose-dependent insulinotropic polypeptide (GIP) receptors, and use the peptide compound. The compound provided by the present invention has an improved agonistic activity at GIP receptors and selectively activate the GIP receptors, and may be used in the manufacture of medicaments associated with type 2 diabetes and weight loss.

### TECHNICAL FIELD

The present invention relates to the field of biomedical technology, and in particular, relates to a novel peptide compound having agonistic activity at glucose-dependent insulinotropic polypeptide (GIP) receptors, and use of the peptide compound.

### BACKGROUND ART

The global prevalence of type 2 diabetes (T2DM) and obesity has continued to increase over the past 30 years. 246 million people worldwide suffer from diabetes. It is estimated that 380 million people will suffer from diabetes by 2025. The increased morbidity and mortality impose a medical demand for more effective treatments.

Incretins are a class of gut hormones in human bodies. Incretins currently identified mainly include two types, namely, glucagon like peptide 1 (GLP-1) and glucose dependent insulinotropic polypeptide (GIP), both of which promote the secretion of insulin and have glucose-dependent hypoglycemic effects. Human GLP-1 is a polypeptide containing 31 amino acids, and is mainly secreted by L cells in the terminal ileum and colon. Human GIP is a polypeptide containing 42 amino acids, and is mainly produced by K cells in the duodenum and jejunum. In clinical trials, after being injected with GIP and GLP-1 in combination, as compared with being injected with GLP-1 alone, a healthy subject not only requires a significantly higher amount of infusion of glucose to develop isoglycaemia, but also has a high level of pancreatic β cells response (Michael, J C E & M, 1993, Vol. 76, No. 4). Therefore, GIP receptor agonists synergistically enhance the hypoglycemic and weight loss effects of GLP-1. In another aspect, GIP receptor agonists may be used as antiemetics or be used to prevent diseases accompanied by vomiting or nausea. The administration of GLP-1 at a high dosage is accompanied by symptoms of vomiting or nausea. Therefore, GIP receptor agonists may be used to inhibit or alleviate symptoms such as vomiting or nausea caused by GLP-1, so as to enhance the pharmaceutical efficacy of GLP-1 at a high dosage.

GIP receptors serve as targets for the treatment of metabolic diseases such as diabetes and obesity, and may be administrated in combination with GLP-1 receptor agonists, or in combination with GLP-1/glucagon receptor co-agonists. Although in prior art patents or products, both GLP-1 and GIP sequences are combined in the same molecule so as to achieve agonistic activities at both GLP-1 receptors and GIP receptors, there are no clear conclusion on the influence of the activation intensity or the activation proportion against GLP-1 and GIP receptors on the pharmaceutical efficacy of the existing molecules. However, once the structure of the molecule is determined, the activation level of the single molecule for the two targets can not be adjusted. There are limitations to achieve the same level of activation of GLP-1 and GIP receptors or to administrate different dosages under the same activation level.

### CONTENTS OF THE INVENTION

A first aspect of the present invention provides a glucose-dependent insulinotropic polypeptide (GIP) analog, wherein amino acids at multiple sites are substituted so that the only lysine modification site in the polypeptide sequence is retained, and the agonistic activity at GIP receptors is maintained. Upon further modification with a long-lasting side chain, the GIP analog may be used in combination with a GLP-1 analog, thereby achieving the above-mentioned objection.

The polypeptide segment of the GIP analog provided by the present invention is:

(a)
YX₂EGTFISDYSIX₁₃X₁₄DX₁₆X₁₇X₁₈QX₂₀X₂₁FVX₂₄WLLAQX₃₀X₃₁;

wherein X₂ is selected from A, D-Ala or Aib, X₁₃ is selected from A or Aib, X₁₄ is selected from M or L, X₁₆ is selected from E, K or R, X₁₇ is selected from I or L, X₁₈ is selected from H or R, X₂₀ is selected from K, E or Q, X₂₁ is selected from D or E, X₂₄ is selected from K or N, X₃₀ is selected from K or R, and X₃₁ is G or is absent; or

(b)
YX₂EGTFISDYSIX₁₃X₁₄DX₁₆X₁₇X₁₈QX₂₀X₂₁FVX₂₄WLLAQX₃₀X₃₁Y₁,

Y₁ may be selected from GPSSGAPPPS, KPSSGAPPPS, GPSSGAPPS, PSSGAPPPS, or PSSGAPPS; or
(c) an amino acid sequence with a sequence identity of 90% or above to (a) or (b), or a polypeptide segment having the function of the polypeptide segment as defined in (a) or (b).

In particular, the amino acid sequences in (c) refer to: the amino acid sequences obtained by substitution, deletion or addition of one or more (specifically, possibly 1-50, 1-30, 1-20, 1-10, 1-5, or 1-3) amino acids from/onto the amino acid sequence as shown in Scheme (a) or (b); or the amino acid sequences obtained by addition of one or more amino acids (specifically, possibly 1-50, 1-30, 1-20, 1-10, 1-5, or 1-3) amino acids at the N-terminus and/or C-terminus; and the polypeptide segments encoded by them have the function of the polypeptide segments as defined in Scheme (a) or (b), respectively.

The GIP-like polypeptide segments of the present invention may have an amino acid sequence as shown in the following table:

| No. | Polypeptide Sequence |
|---|---|
| hGIP (1-42) | YAEGTFISDYSIAMDKIHQQDFVNWLLAQKGKK |
| | NDWKHNITQ |
| Sequence 1 (Seq ID. 1) | YAibEGTFISDYSIAibLDKIHQEDFVKWLLAQKG |
| Sequence 2 (Seq ID. 2) | YAibEGTFISDYSIAibLDEIHQKDFVKWLLAQKG |
| Sequence 3 (Seq ID. 3) | YAibEGTFISDYSIALDKIHQEDFVKWLLAQKG |
| Sequence 4 (Seq ID. 4) | YAibEGTFISDYSIALDEIHQKDFVKWLLAQKG |
| Sequence 5 (Seq ID. 5) | YAibEGTFISDYSIALDEIHQKDFVNWLLAQRG |
| Sequence 6 (Seq ID. 6) | YAibEGTFISDYSIALDELHQKDFVNWLLAQRG |
| Sequence 7 (Seq ID. 7) | YAibEGTFISDYSIALDKIHQQDFVNWLLAQRG |
| Sequence 8 (Seq ID. 8) | YAibEGTFISDYSIALDKLHQQDFVNWLLAQRG |
| Sequence 9 (Seq ID. 9) | YAibEGTFISDYSIALDKIHQEDFVNWLLAQRG |
| Sequence 10 (Seq ID. 10) | YAibEGTFISDYSIALDKLHQEDFVNWLLAQRG |
| Sequence 11 (Seq ID. 11) | YAibEGTFISDYSIALDRIHQEDFVKWLLAQRG |
| Sequence 12 (Seq ID. 12) | YAibEGTFISDYSIALDRIHQEDFVNWLLAQKG |
| Sequence 13 (Seq ID. 13) | YAibEGTFISDYSIALDRIHQQDFVKWLLAQRG |
| Sequence 14 (Seq ID. 14) | YAibEGTFISDYSIALDRIHQQDFVNWLLAQKG |
| Sequence 15 (Seq ID. 15) | YAibEGTFISDYSIALDRLHQEDFVKWLLAQRG |
| Sequence 16 (Seq ID. 16) | YAibEGTFISDYSIALDRLHQEDFVNWLLAQKG |
| Sequence 17 (Seq ID. 17) | YAibEGTFISDYSIALDRLHQQDFVKWLLAQRG |
| Sequence 18 (Seq ID. 18) | YAibEGTFISDYSIALDRLHQQDFVNWLLAQKG |

The GIP polypeptide analogs according to the present invention are artificially designed and can generally be derived from naturally occuring GIP (hGIP (1-42)).

The present invention provides a long-acting conjugate of GIP analog, consisted of the GIP analog and a long-acting conjugating component, wherein the long-acting conjugating component is connected to the GIP analog; and the long-acting conjugating component is selected from the group consisting of fatty acid side chains, polymers, cholesterols, albumins and segments thereof, albumin-binding substances, polymers of repeating units having specific amino acid sequences, antibodies, antibody segments, FcRn-binding substances, in vivo connective tissues, nucleotides, fibronectin, transferrin, saccharides, heparin, and elastin.

The long-acting conjugate of GIP polypeptide analog provided by the present invention may further comprise a side chain modification. The side chain modification can extend the half-life of GIP polypeptide analog in vivo. The side chain modification includes, but is not limited to one or more of fatty acids, aliphatic chains, polyethylene glycols (PEGs) and other structures. The active group of the side chain modification (active groups such as carboxylic acid groups from the aliphatic chains or PEGs, or a maleimide group, etc.) may react with the amino acid residue of the GIP-like polypeptide segment so as to undergo various types of condensation reactions. There may also be a linker between the GIP-like polypeptide segment and the side chain modification. The linker may react with the amino acid residue on the GIP-like polypeptide segment and the active group on the side chain modification, respectively. The linker includes, but is not limited to the following structures, for example, -OEG-(-2-(2-(2-aminoethoxy)ethoxy)acetyl-), -2×OEG-, -γGlu-(-γ-glutamyl-), -β-alanyl-, -L-2-aminobutyryl-, -ε-aminocaproyl-, -D-γ-glutamyl- or its dipeptide, -β-Ala-β-Ala-, -γGlu-γGlu-, -5-aminovaleryl-, -ω-aminooctanoyl-, -9-aminononanoyl-, -10-amino-n-decanoyl-, -γGlu-OEG-, -γGlu-2×OEG-, -D-γGlu-2×OEG-, -2×OEG-γGlu-, -γGlu-3×OEG-γGlu-8×PEG-(-3-((γ-glutamine)-8×PEG)-propionyl-).

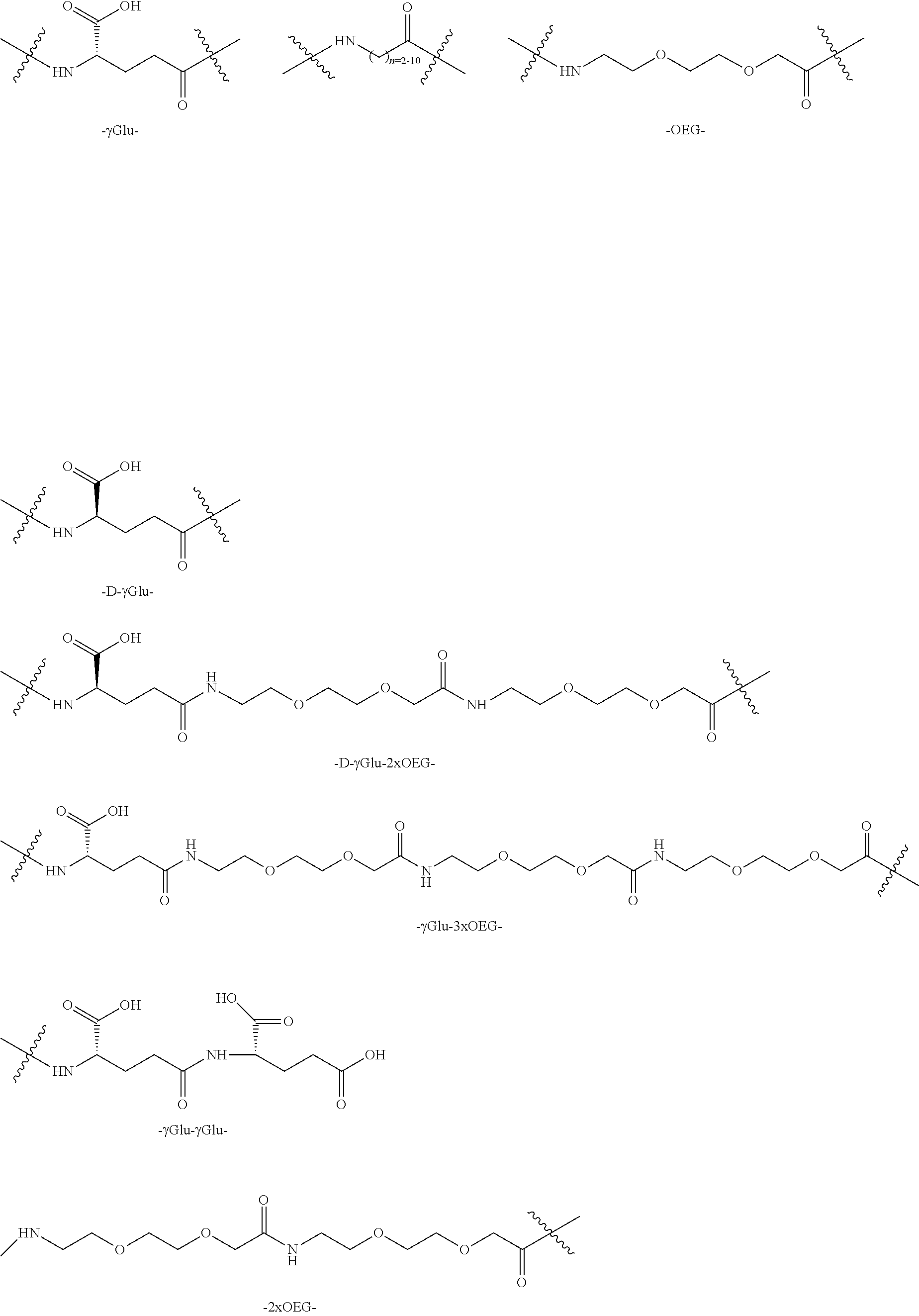

The fatty acid or aliphatic chain structure includes, but is not limited to linear fatty acid chain. For example, the fatty acid may be a C₈-C₃₀, C₈-C₂₀, or C₁₂-C₂₀ fatty acid. The fatty acid may be a monocarboxylic acid and/or a dicarboxylic acid. The fatty acid may be linear or branched. The fatty acid or aliphatic chain may be combined with the above-mentioned linker structure to form a side chain modification. The side chain modification structure may be selected from, but is not limited to the following structures:

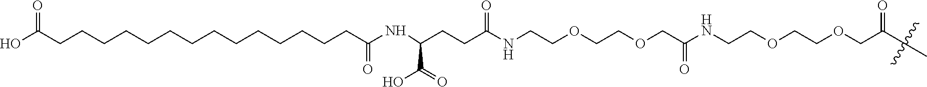 [SMILES: *C(=O)COCCOCCNC(=O)COCCOCCNC(=O)CC[C@H](NC(=O)CCCCCCCCCCCCCCC(=O)O)C(=O)O]

16-{[(23S)-23-carboxyl-2,11,20-trioxo-10,19-diaza-4,7,13,16-tetraoxatricosyl-23-yl]amino}-16-oxohexadecanoic acid ethane

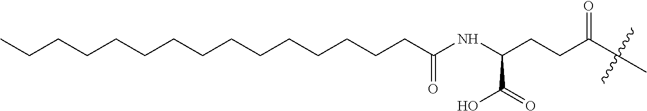 [SMILES: *C(=O)CC[C@H](NC(=O)CCCCCCCCCCCCCCC)C(=O)O]

(2S)-5-oxo-2-[(1-oxohexadecyl)amino]hexanoic acid ethane

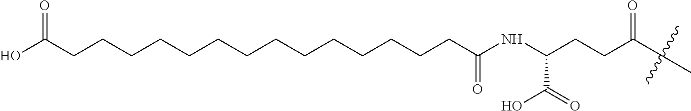 [SMILES: *C(=O)CC[C@@H](NC(=O)CCCCCCCCCCCCCCC(=O)O)C(=O)O]

16-{[(1R)-1-carboxyl-4-oxopentyl]amino}-16-oxohexadecanoic acid ethane

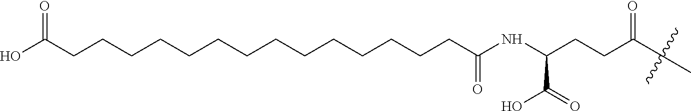 [SMILES: *C(=O)CC[C@H](NC(=O)CCCCCCCCCCCCCCC(=O)O)C(=O)O]

16-{[(1S)-1-carboxyl-4-oxopentyl]amino}-16-oxohexadecanoic acid ethane

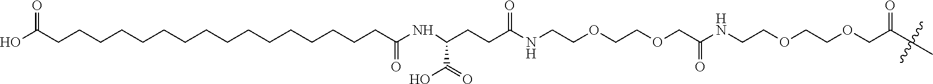 [SMILES: *C(=O)COCCOCCNC(=O)COCCOCCNC(=O)CC[C@@H](NC(=O)CCCCCCCCCCCCCCCCC(=O)O)C(=O)O]

18-{[(23R)-23-carboxyl-2,11,20-trioxo-10,19-diaza-4,7,13,16-tetraoxatricosyl-23-yl]amino}-18-oxooctadecanoic acid ethane

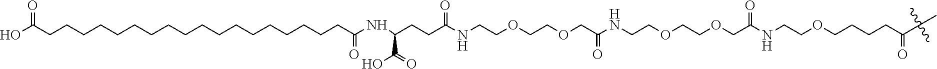 [SMILES: *C(=O)CCCCOCCNC(=O)COCCOCCNC(=O)COCCOCCNC(=O)CC[C@H](NC(=O)CCCCCCCCCCCCCCCCCCC(=O)O)C(=O)O]

20-{[(32S)-32-carboxyl-2,11,20,29-tetraoxo-7,13,16,22,25-pentaoxa-10,19,28-triazatridodecyl-32-yl]amino}-20-oxoeicosanoic acid ethane

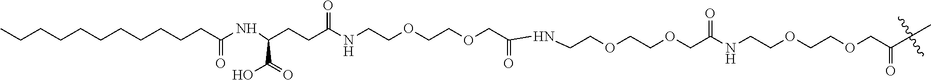 [SMILES: *C(=O)COCCOCCNC(=O)COCCOCCNC(=O)COCCOCCNC(=O)CC[C@H](NC(=O)CCCCCCCCCCC)C(=O)O]

(2S)-5-[(8,26-dioxo-9,18-diaza-3,6,12,15,21,24-hexaoxaheptacosane-1-yl)amino]-2-[(1-oxododecyl)amino]pentanoic acid ethane

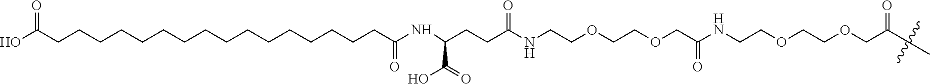 [SMILES: *C(=O)COCCOCCNC(=O)COCCOCCNC(=O)CC[C@H](NC(=O)CCCCCCCCCCCCCCCCC(=O)O)C(=O)O]

18-{[(23S)-23-carboxyl-2,11,20-trioxo-10,19-diaza-4,7,13,16-tetraoxatricosyl-23-yl]amino}-18-oxooctadecanoic acid ethane

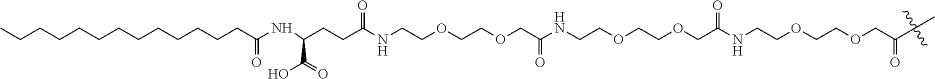 [SMILES: *C(=O)COCCOCCNC(=O)COCCOCCNC(=O)COCCOCCNC(=O)CC[C@H](NC(=O)CCCCCCCCCCCCC)C(=O)O]

(2S)-5-oxo-2-[(1-oxotetradecyl)amino]-5-[(8,17,26-trioxo-9,18-diaza-3,6,12,15,21,24-hexaoxaheptacosane-1-yl)amino]pentanoic acid ethane

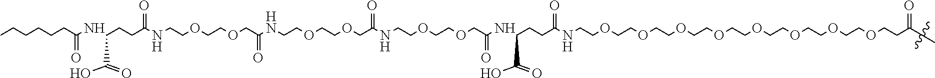 [SMILES: *C(=O)CCOCCOCCOCCOCCOCCOCCOCCOCCNC(=O)CC[C@H](NC(=O)COCCOCCNC(=O)COCCOCCNC(=O)COCCOCCNC(=O)CC[C@@H](NC(=O)CCCCCC)C(=O)O)C(=O)O]

(2R)-5-{[(28S)-28-carboxyl-8,17,26,31,59-pentaoxo-9,18,27,32-tetraza-3,6,12,15,21,24,35,38,41,44,47,50,53,56-tetradecaoxahexacontyl-1-yl]amino}-5-oxo-2-[(1-oxoheptyl)amino]pentanoic acid ethane

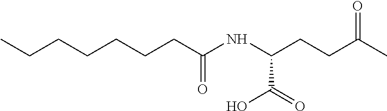 [SMILES: CCCCCCCC(=O)N[C@H](CCC(C)=O)C(=O)O]

(2R)-5-oxo-2-[(1-oxooctyl)amino]hexanoic acid

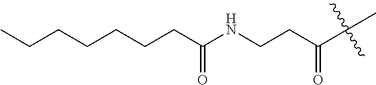 [SMILES: *C(=O)CCNC(=O)CCCCCCC]

N-(3-oxobutyl)octanamide ethane

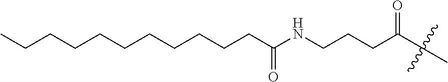 [SMILES: *C(=O)CCCNC(=O)CCCCCCCCCCC]

N-(4-oxopentyl)dodecanamide ethane

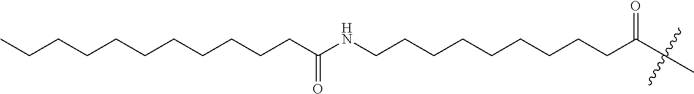 [SMILES: *C(=O)CCCCCCCCCNC(=O)CCCCCCCCCCC]

N-(10-oxoundecyl)dodecanamide ethane

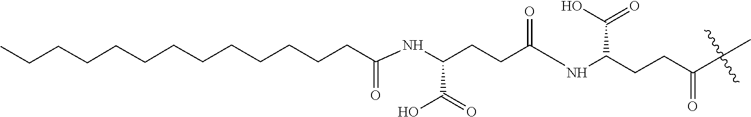

2-{[(4R)-4-carboxyl-1-oxo-4-[(1-oxotetradecyl)amino]butyl]amino}-5-oxohexanoic acid

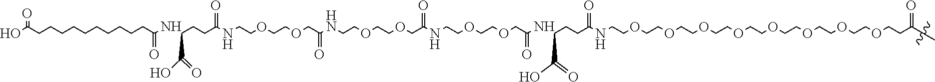 [SMILES: *C(=O)CCOCCOCCOCCOCCOCCOCCOCCOCCNC(=O)CC[C@H](NC(=O)COCCOCCNC(=O)COCCOCCNC(=O)COCCOCCNC(=O)CC[C@H](NC(=O)CCCCCCCCCCC(=O)O)C(=O)O)C(=O)O]

11-{[(33S,65S)-33,65-dicarboxyl-2,30,35,44,53,62-hexaoxo-29,34,43,52,61-pentaza-5,8,11,14,17,20,23,26,37,40,46,49,55,58-tetradecaoxapentahexacontane-65-yl]amino}-11-oxoundecane-1-carboxylic acid ethane

The side chain modification structure is preferably linked to the polypeptide via a lysine in the GIP polypeptide analog.

The long-acting conjugate of GIP analog provided by the present invention may be:

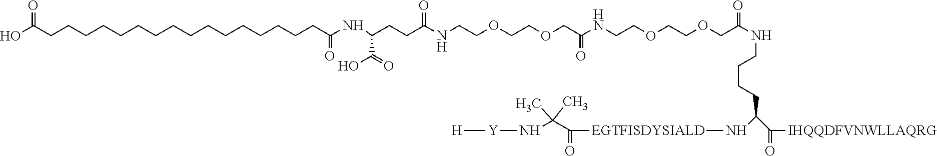

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R30]-hGIP(1-31);

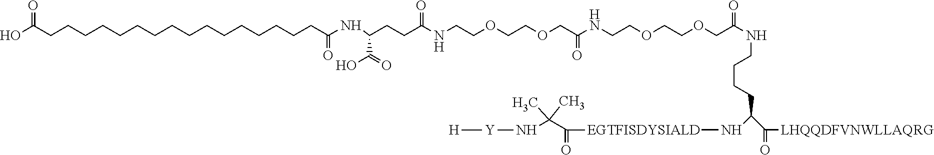

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, R30]-hGIP(1-31);

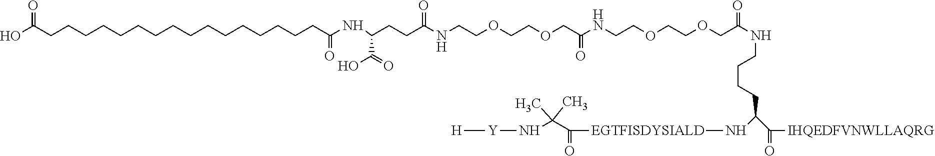

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, E20, R30]-hGIP(1-31);

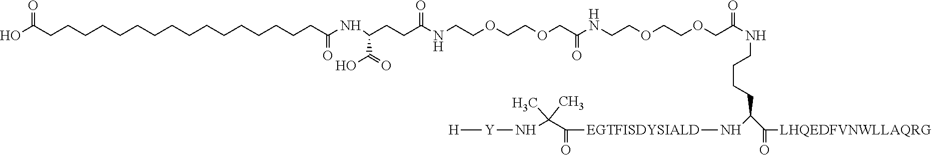

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, E20, R30]-hGIP(1-31); and

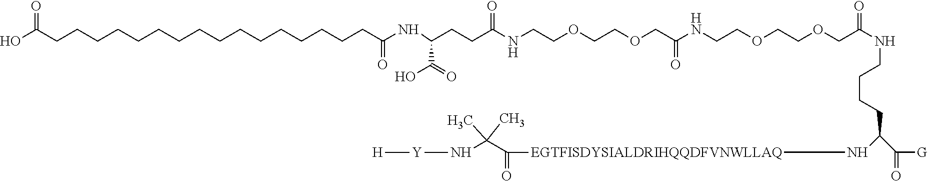

N-{ε-30}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R16]-hGIP(1-31).

A second aspect of the present invention provides an isolated polynucleotide encoding the above-mentioned GIP analog.

A third aspect of the present invention provides a recombinant expression vector comprising the isolated polynucleotide provided by the second aspect of the present invention.

A fourth aspect of the present invention provides a host cell comprising the recombinant expression vector provided by the third aspect of the present invention or having the isolated polynucleotide provided by the second aspect of the present invention integrated into the genome thereof.

A fifth aspect of the present invention provides a method for preparing the GIP analog provided by the first aspect of the present invention, comprising preparing said GIP analog by a chemical synthesis method, for example, by a standard solid-phase or liquid-phase process, either stepwise or by segment assembly, and isolating and purifying the final GIP analog product. The preparation method may further comprise cultivating the host cell provided by the fourth aspect of the present invention, so that the cell expresses the segment of the GIP analog; isolating and purifying the host cell to obtain the GIP analog; followed by chemically cross-linking the long-acting side chain modification to the GIP analog to prepare the long-acting conjugate of GIP analog.

A sixth aspect of the present invention provides use of the GIP analog in the manufacture of a medicament for the treatment and/or prevention of metabolic diseases. The metabolic diseases may be diabetes-related diseases, or obesity or obesity-related diseases, and liver diseases, and in particular may be selected from diabetes, obesity, dyslipidemia, non-alcoholic fatty liver disease (NAFLD)/non-alcoholic steatohepatitis (NASH), other diabetes-related metabolic syndromes and the like.

The diabetes-related diseases include insulin resistance, glucose intolerance, elevated fasting glucose, prediabetes, type 1 diabetes, type 2 diabetes, gestational diabetes hypertension, dyslipidemia, and combinations thereof.

The diabetes-related diseases further include atherosclerosis, arteriosclerosis, coronary heart diseases, peripheral arterial diseases, and stroke; or dyslipidemia, elevated blood pressure, hypertension, prothrombotic states, bone-related disorders, proinflammatory states, and atherogenic dyslipidemia related diseases.

The compounds according to the present invention may also be used as an excipient agent for weight management in patients with obesity, and may also be used for obesity-related diseases, including obesity-related inflammation, obesity-related gallbladder diseases, and obesity-induced sleep apnoea. In addition, the compounds may be used in the treatment of Alzheimer's disease (AD).

The seventh aspect of the present invention provides a pharmaceutical composition comprising a therapeutically effective amount of the GIP analog and the long-acting conjugate thereof provided in the first aspect of the present invention. The GIP receptor agonist compounds or salts or solvates thereof according to the present invention may be formulated into a pharmaceutical composition ready for storage or administration, which generally comprises commonly used pharmaceutical carriers. In some embodiments, the pharmaceutical composition is formulated into a liquid suitable for injection or infusion, or formulated into a formulation which slowly releases the GIP analogue, or formulated into a solid oral formulation.

The GIP analogs provided in the present invention may be administered in combination with at least one other agent useful in treating diabetes, obesity, dyslipidemia or hypertension. The at least two active agents may be administered together or separately, either as a part of the same pharmaceutical formulation or as different formulations. The GIP analogs and long-acting conjugates thereof (or salts or solvates thereof) according to the present invention may be used in combination with an anti-diabetic agent or anti-obesity agent, including but not limited to metformin, sulfonylureas, glinides, DPP-IV inhibitors, glitazones, insulin, GLP-1 receptor agonists, or GLP-1/glucagon receptor co-agonists, preferably GLP-1 receptor agonists, preferably Semaglutide.

### Definitions

GLP-1 receptor agonist: “GLP-1 receptor agonist” may be defined as polypeptides, proteins or other small molecules which bind to GLP-1 receptor (GLP-1R) and are capable of triggering the same or similar characteristic responses as naturally occurring GLP-1 does. GLP-1R agonists fully or partially activate GLP-IR, which in turn triggers a series of intracellular downstream signaling pathway responses, resulting in corresponding cell activity, such as insulin secretion from B-cells.

GIP receptor agonist: GIP is a 42-amino acid polypeptide released from intestinal K cells after ingestion of food. The main function of GIP is to inhibit secretion of gastric acid and enhance glucose-stimulated insulin secretion. GIP receptor agonists may be defined as polypeptides, proteins, or other small molecules that are capable of binding to a GIP receptor (GIPR) and activating signal transduction through the receptor, for example, by producing CAMP or inducing Ca²⁺ release. Thus, the agonist activity at GIP receptors may be measured by evaluating GIP receptor signal transduction, which may be measured by, for example, cAMP production or Ca²⁺ release.

GIP analogs: GIP analogs are highly similar to human GIP, and can lower blood glucose in a glucose concentration-dependent manner. Meanwhile, GIP analogs also have multiple effects similar to that of human GIP. Polypeptide analogs with properties superior to those of naturally occurring GIP are obtained after introducing mutation or conducting reconstruction at site(s) on the naturally occurring GIP.

Glucagon receptor (GCGR) agonists: Glucagon receptor (GCGR) agonists may be defined as polypeptides, proteins or other small molecules which bind to GCGR and are capable of triggering the same or similar characteristic responses as naturally occurring glucagon. GCGR agonists fully or partially activate GCGR, which in turn trigger a series of intracellular downstream signaling pathway responses, resulting in corresponding cell activities, such as hepatocyte glycogenolysis, gluconeogenesis, fatty acid oxidation, ketogenesis and the like.

GLP-1R/GIPR Co-agonist: The GLP-1R/GIPR dual-effect agonists of the present invention include proteins or peptides that have both GLP-IR and GIPR agonistic activities.

The amino acid sequences of the present invention contain the standard one-letter or three-letter codes for twenty natural amino acids. In addition, “Aib” is α-aminoisobutyric acid.

Side Chain Modifications: side chain modifications can bind to plasma proteins (for example, albumin) in the blood stream, thereby protecting the compounds according to the invention from enzymatic degradation and renal clearance, thereby extending the half-lives of the compounds. The substituents can also modulate the efficacies of the compounds.

EC50 (concentration for 50% of maximal effect): EC50 generally refers to the concentration of an agent or substance required for stimulating 50% of its corresponding biological response. It is shown that the lower the EC50 value is, the stronger the stimulatory or agonistic ability of the agent or substance is. This may be more intuitively reflected for example as the stronger the resultant intracellular signal is, the better ability to induce the production of a hormone.

Pharmaceutically Acceptable Salts: “pharmaceutically acceptable salts” refer to salts of a compound, including pharmaceutically acceptable salts, for example, acid addition salts and basic salts. Examples of acid addition salts include a hydrochloride, citrate and acetate. Examples of basic salts include salts in which the cation is selected from alkali metals, such as sodium and potassium; alkaline earth metals, such as calcium; and ammonium ions N⁺(R³)₃(R⁴), wherein R³ and R⁴ are independently hydrogen, optionally substituted C₁₋₆ alkyl, optionally substituted C₂₋₆ alkenyl, optionally substituted aryl or optionally substituted heteroaryl.

### DESCRIPTION OF FIGURES

FIG. 1 illustrates the blood glucose change value relative to 0 min for long-acting conjugates of GIP analogs in the IPGTT (Intraperitoneal Glucose Tolerance Test) of Example 8.

FIG. 2 illustrates the area under the curve of the blood glucose change value for long-acting conjugates of GIP analogs in the IPGTT of Example 8.

FIG. 3 illustrates the area under the curve of the blood glucose change value for the combination of long-acting conjugates of GIP analogs and GLP-1 analogs in the IPGTT of Example 10.

FIG. 4 illustrates the effect of the combination of long-acting conjugates of GIP analogs and a constant amount of GLP-1 analogs on the body weight change of DIO mice in Example 11.

FIG. 5 illustrates the effect of the combination of a constant amount of long-acting conjugates of GIP analogs and GLP-1 analogs on the body weight change of DIO mice in Example 11.

FIG. 6 illustrates the effect of the combination of long-acting conjugates of GIP analogs and GLP-1 analogs on the water intake change of DIO mice in Example 13.

FIG. 7 illustrates the effect of the combination of long-acting conjugates of GIP analogs and GLP-1 analogs on the cumulative water intake change of DIO mice in Example 13.

### DETAILED DESCRIPTION OF EMBODIMENTS

Embodiment 1. A glucose-dependent insulinotropic polypeptide (GIP) analog, comprising a GIP-like polypeptide segment, wherein the GIP-like polypeptide segment is:
(a) YX₂EGTFISDYSIX₁₃X₁₄DX₁₆X₁₇X₁₈QX₂₀X₂₁FVX₂₄WLLAQX₃₀X₃₁;
wherein X₂ is selected from A, D-Ala or Aib, X₁₃ is selected from A or Aib, X₁₄ is selected from M or L, X₁₆ is selected from E, K or R, X₁₇ is selected from I or L, X₁₈ is selected from H or R, X₂₀ is selected from K, E or Q, X₂₁ is selected from D or E, X₂₄ is selected from K or N, X₃₀ is selected from K or R, and X₃₁ is G or is absent; or

(b) YX₂EGTFISDYSIX₁₃X₁₄DX₁₆X₁₇X₁₈QX₂₀X₂₁FVX₂₄WLLAQX₃₀X₃₁Y₁,
Y₁ may be selected from GPSSGAPPPS, KPSSGAPPPS, GPSSGAPPS, PSSGAPPPS, or PSSGAPPS; or
(c) an amino acid sequence with a sequence identity of 90% or above to (a) or (b), or a polypeptide segment having the function of the polypeptide segment as defined in (a) or (b).

Embodiment 2. The GIP analog according to Embodiment 1, comprising an amino acid sequence selected from SEQ ID NOs: 1 to 18.

Embodiment 3. A long-acting conjugate of GIP analog, characterized in that it is consisted of the GIP analog according to any one of Embodiment 1 or 2 and a long-acting conjugating component.

Embodiment 4. The long-acting conjugate of GIP analog according to Embodiment 3, characterized in that the long-acting conjugating component is connected to the GIP analog; and the long-acting conjugating component is selected from the group consisting of fatty acid side chains, polymers, cholesterols, albumins and segments thereof, albumin-binding substances, polymers of repeating units having specific amino acid sequences, antibodies, antibody segments, FcRn-binding substances, in vivo connective tissues, nucleotides, fibronectin, transferrin, saccharides, heparin, and elastin.

Embodiment 5. The long-acting conjugate of GIP analog according to Embodiment 4, characterized in that the fatty acid side chain is selected from C₈ to C₂₆ fatty acids, the fatty acid side chain is a linear or branched chain, and the fatty acid side chain is a monocarboxylic acid and/or a dicarboxylic acid.

Embodiment 6. The long-acting conjugate of GIP analog according to Embodiment 5, characterized in that the fatty acid side chain is selected from HOOC(CH₂)₁₄CO—, HOOC(CH₂)₁₅CO—, HOOC(CH₂)₁₆CO—, HOOC(CH₂)₁₇CO—, HOOC(CH₂)₁₈CO—, HOOC(CH₂)₁₉CO—, HOOC(CH₂)₂₀CO—, HOOC(CH₂)₂₁CO— or HOOC(CH₂)₂₂CO—.

Embodiment 7. The long-acting conjugate of GIP analog according to Embodiment 6, characterized in that it is selected from the group consisting of:

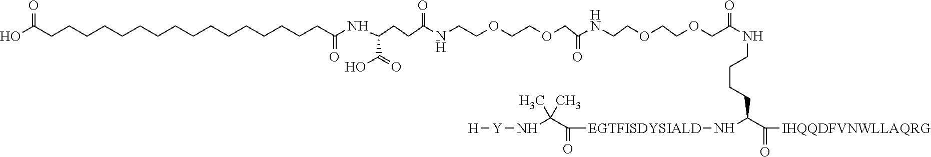

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R30]-hGIP(1-31);

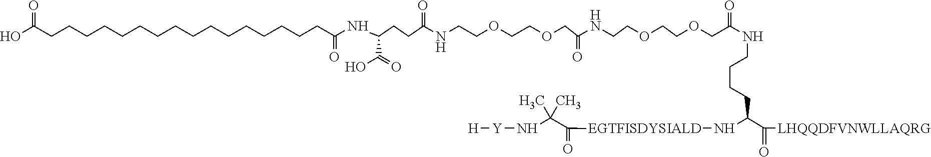

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, R30]-hGIP(1-31);

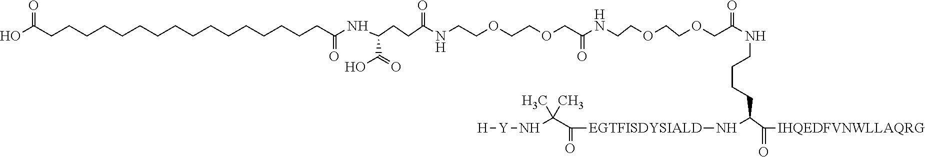

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, E20, R30]-hGIP(1-31);

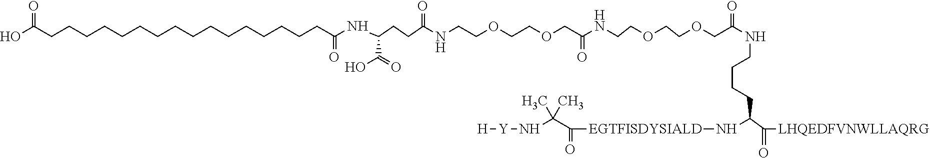

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, E20, R30]-hGIP(1-31); and

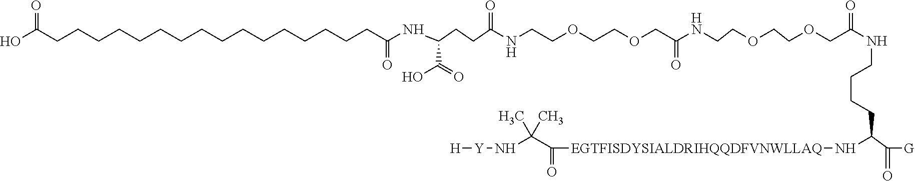

N-{ε-30}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R16]-hGIP(1-31).

Embodiment 8. The GIP analog or long-acting conjugate thereof according to any one of Embodiments 1 to 7, characterized in that it has agonistic activity at GIP receptors, but has no agonistic activity at GLP-1 receptors.

Embodiment 9. An isolated polynucleotide encoding the GIP analog according to any one of Embodiment 1 or 2.

Embodiment 10. A recombinant expression vector comprising the isolated polynucleotide according to Embodiment 9.

Embodiment 11. A host cell comprising the recombinant expression vector according to Embodiment 10, or having the isolated polynucleotide according to Embodiment 9 integrated into the genome thereof.

Embodiment 12. A method for preparing a GIP analog, comprising: (1) synthesizing the GIP analog according to any one of Embodiment 1 or 2 by a chemical synthesis method; or (2) cultivating the host cell according to Embodiment 11, so that the cell expresses the GIP analog according to any one of Embodiment 1 or 2, and subsequently isolating and purifying the host cell to obtain the GIP analog; or preparing the GIP analog by both of method (1) and method (2)

Embodiment 13. A method for preparing a long-acting conjugate of a GIP analog, comprising: chemically cross-linking the long-acting conjugating component defined in any one of Embodiments 4 to 6 to the GIP analog defined in Embodiment 12 by a chemical synthesis method.

Embodiment 14. The GIP analog or the long-acting conjugate thereof according to any one of Embodiments 1 to 8 useful in the manufacture of a medicament for the treatment and/or prevention of weight management, obesity and obesity-related diseases.

Embodiment 15. The GIP analogue or the long-acting conjugate thereof according to any one of Embodiments 1 to 8 useful in the manufacture of a medicament for the treatment and/or prevention of all types of diabetes such as type 2 diabetes, and diabetes-related diseases.

Embodiment 16. A pharmaceutical composition, comprising the GIP analog or the long-acting conjugate thereof according to any one of Embodiments 1 to 8, and at least one pharmaceutically acceptable excipient.

17. The pharmaceutical composition according to Embodiment 16, characterized in that the composition is formulated into a liquid formulation suitable for injection or infusion, or an oral solid formulation.

18. The pharmaceutical composition according to Embodiment 17, characterized in that the composition is formulated into a formulation which slowly releases the GIP analogue or the long-acting conjugate thereof in the composition.

19. The pharmaceutical composition according to any one of Embodiments 16 to 18, further comprising a GLP-1 receptor agonist, a glucagon receptor agonist, and a GLP-1/glucagon receptor coagonist.

20. The pharmaceutical composition according to any one of Embodiments 16 to 19 useful in the manufacture of a medicament for the treatment and/or prevention of weight management, obesity and obesity-related diseases.

21. The pharmaceutical composition according to any one of Embodiments 16 to 19 useful in the manufacture of a medicament for the treatment and/or prevention of all types of diabetes such as type 2 diabetes, and diabetes-related diseases.

### MODE OF CARRYING OUT THE INVENTION

Unless otherwise specified below, all technical and scientific terms recited in the present invention have the meaning commonly understood by those skilled in the art to which the present invention belongs.

The methods for synthesizing the compounds according to the present invention are well-known in the art. The compounds of the present invention may be produced by recombinant methods, i.e., bonding a coding nucleic acid to a functional control sequence that directs its expression, culturing the host cells in a suitable culture media so as to obtain the expressed polypeptide, and performing chemical modifications thereon to obtain the compounds of the present invention. Host cells may be selected from Escherichia coli, Saccharomyces cerevisiae, and mammalian BHK or CHO cell lines.

The compound according to the present invention can also be produced by standard peptide synthesis methods, for example, by means of standard solid-phase or liquid-phase process, either stepwise or by segment assembly, and isolating and purifying the final GIP analog product; or may be prepared by any combination of a recombinant method and a synthesis method.

### Example 1: Preparation of Polypeptide Having Sequence 3

The nucleotide sequence (E3 being the starting point) GAAGGCACCT TCATCTCCGA TTACTCCATC GCGCTGGACA AAATCCACCA GGAAGATTTC GTAAAATGGC TGCTGGCGCA GAAAGGC corresponding to the amino acid sequence represented by SEQ ID NO:3 was synthesized artificially (synthesized by Nanjing Tsingke Biotechnology Co. Ltd.). Double digestion was conducted with NcoI and XhoI on the synthesized gene and pET28a plasmid vector (commercially available from Nanjing Tsingke Biotechnology Co., Ltd.). The digested product was gel extracted. T4 was used for ligating the digested product. The ligated product was transformed to a BL21(DE3) host Escherichia coli (commecially avaliable from Nanjing Tsingke Biotechnology Co., Ltd.). A single colony was picked and seeded into a test tube containing LB liquid culture medium, and incubated at 37° C. and 250 rpm for 6 to 10 hours. The recombinant plasmid was extracted and digested for verification. The recombinant plasmid demonstrating correct insertion of genes was sequenced (the sequencing was conducted by Nanjing Tsingke Biotechnology Co., Ltd.). The strains with correctly inserted genes were screened as genetically engineered strains expressing the amino acids shown in SEQ ID NO:1.

The screened genetically engineered strains were seeded into an LB plate containing 80 mg/L Kanamycin at 37ºC for 14 to 18 hours. A single colony was picked into an LB culture medium containing 80 mg/L kanamycin, and incubated at 37° C. and 250 rpm for 10-15 hours to form a seed liquid. The seed liquid was transferred into an LB culture medium containing 80 mg/L kanamycin at an inoculum concentration of 5%, and incubated at 37° C. and 250 rpm until the OD (600 nm) was 1.5. IPTG was added at a final concentration of 0.3 mmol/L. Fermentation was initiated at 23° C. After 5 hours, the strains were collected by centrifugation, re-suspended in a buffer, and ultrasonically crushed. The supernatant was collected by centrifugation. The supernatant was purified on a nickel ion chelation affinity chromatography column (commecially avaliable from Nanjing Tsingke Biotechnology Co., Ltd.). Protein elution peaks were collected, and purification of the proteins was detected by SDS-PAGE. The corresponding fractions were collected, ultrafiltrated and desalted to harvest the fusion protein. The fusion protein was digested with Enterokinase (Ekase) to obtain the respective polypeptide. YAib was chemically coupled with the obtained polypeptide to obtain the polypeptide having Sequence 3: YAibEGTFISDYSIALDKIHQEDFVKWLLAQKG. The polypeptides having the remaining sequences were prepared according to the process for the preparation of the polypeptide having Sequence 3.

### Example 2: Preparation of Long-Acting Conjugate of Sequence 7 (Peptide 7)

The meanings of abbreviations in Examples 2-6 are as follows.

Fmoc: 9-fluorenylmethoxycarbonyl; HOBt: 1-hydroxybenzotriazole; DMAP: dimethylaminopyridine; DIC: diisopropylcarbodiimide, DMF: N,N-dimethylformamide, Boc: tert-butoxycarbonyl; tBu: tert-butyl; DCM: dichloromethane; and MeOH: methanol.

The following amino acids were used in the synthesis for extending the polypeptides: Fmoc-L-Tyr(tBu)-OH; Fmoc-L-Aib-OH; Fmoc-LGlu(OtBu)-OH; Fmoc-L-Gly-OH; Fmoc-L-Thr(tBu)-OH; Fmoc-L-Phe-OH; Fmoc-L-Ile-OH; Fmoc-L-Ser(tBu)-OH; Fmoc-L-Asp(OtBu)-OH; Fmoc-L-Tyr(tBu)-OH; Fmoc-L-Ser(tBu)-OH; Fmoc-L-Ile-OH; Fmoc-Ala-OH; Fmoc-L-Leu-OH; F-moc-L-Asp(OtBu)-OH; F-moc-L-Lys(Mtt)-OH; Fmoc-L-Ile-OH; Fmoc-L-His(Trt)-OH; Fmoc-L-Gln(Trt)-OH; Fmoc-L-Gln(Trt)-OH; F-moc-L-Asp(OtBu)-OH; Fmoc-L-Phe-OH; Fmoc-L-Val-OH; Fmoc-L-Asn(Trt)-OH; Fmoc-L-Trp(Boc)-OH; Fmoc-L-Leu-OH; Fmoc-L-Leu-OH; Fmoc-Ala-OH; Fmoc-L-Gln(Trt)-OH; Fmoc-Arg(Pbf)-OH; and Fmoc-L-Gly-OH.

### Synthesis of Fmoc-L-Tyr(tBu)-Wang Resin:

25.86 g Wang resin with a substitution degree of 0.58 mmol/g was weighed and added to a solid-phase reaction column. After 200 mL DCM was added to swell the resin for 30 minutes, the resin was washed with DMF for three times, 200 mL each time. Separately, 13.85 g Fmoc-L-Tyr(tBu)-OH, 6.86 g HOBt and 0.28 g DMAP were then dissolved in DMF and activated by adding 5.2 mL DIC at 5 to 8ºC for 5 minutes, and then loaded into the above-mentioned reaction column loaded with resin. The reaction was conducted for 16 hours. After the Kaiser assay showed negative, the resin was washed with DMF 2 times, with MeOH 2 times, with DCM 2 times and with MeOH 2 times, and 200 mL solvent each time. The resin was collected and dried under reduced pressure at room temperature to obtain 28.56 unblocked Fmoc-L-Tyr(tBu)-Wang resin.

The above-mentioned resin was added into the reaction column, and 200 mL DCM was added to swell the resin for 30 minutes. DCM was drawn to dryness, and the resin was washed with DMF 3 times, 200 mL each time. 200 mL DMF and 26 mL blocking solution (the blocking solution is tacetic anhydride and pyridine wherein the ratio of acetic anhydride to pyridine by volume is 1:1) was further added to the reaction column. The reaction was conducted for 2 hours. The resin was sequentially washed with DMF 2 times, with MeOH 2 times, with DCM 2 times and with MeOH 2 times, and 200 mL washing solvent each time. The resin was collected and dried under reduced pressure at room temperature to obtain 32.02 g Fmoc-Tyr(tBu)-Wang resin.

### Synthesis of Peptide Resin:

The above-mentioned blocked F-moc-L-Tyr(tBu)-Wang resin (1.0 mmol) was added to the reaction column. The resin was swelled with 20 mL DCM for 30 minutes, and then washed with DMF for three times, 20 mL each time. After the washing was completed, 10 mL DBLK solution (20% piperidine/DMF (V/V)) was added to the reaction column. The reaction was conducted for 5 minutes. The resin was suction filtered, and washed with 20 mL DMF once. Another 10 mL DBLK solution (20% piperidine/DMF (V/V)) was added, and the reaction was continued for 10 minutes. A sample was taken for intermediate control until the Kaiser assay is positive. The resin was suction filtered, and washed with DMF three times, 20 mL each time. Separately, Fmoc-L-Aib-OH (1.62 g, 5.0 eq) and HOBt (0.81 g, 6.0 eq) were added to 10 mL DMF and dissolved. The resultant mixture was activated by adding DIC (0.69 g, 5.5 eq) at 5 to 8ºC for 5 minutes, and then added to the reaction column. The reaction was conducted for 1 hour. A sample was taken and measured in a Kaiser assay. The result is negative, that is, the reaction was complete. The resin was washed with DMF for three times, 20 mL each time. The above deprotection and coupling operations were repeated to complete the coupling of other amino acids in sequence according to the polypeptide sequence. When F-moc-L-Lys(Mtt)-OH is coupled, the resin was washed with 70% HFIP+3% TIS in DCM twice, 0.5 hour each time. After washing the resin, DMF and DCM were used for washing so as to remove the Mtt protection group. And then, (S)-22-tert-butoxycarbonyl-43,43-dimethyl-10,19,24,41-tetraoxo-3,6,12,15,42-pentaoxa-9,18,23-triazatetracycloheptanoic acid was added. The procedure was continued by using suitable amino acid groups step by step until the final amino acid was coupled. The protection groups were removed according the method mentioned above. After deprotection was complete, the resin was washed with DMF 2 times, with MeOH 2 times, with DCM 2 times and with MeOH 2 times, 20 mL washing solvent each time. The material was collected and dried under reduced pressure at room temperature to obtain 13.06 g of the target peptide resin.

### Cleavage of Crude Peptide:

5.01 g of the above-mentioned peptide resin was weighed and slowly added to 50 mL lysis solution (trifluoroacetic acid:thioanisole:anisole:ethanedithiol=90:5:3:2) at 20-30° C. After the addition was completed, the reaction was conducted for 2 hours. After the reaction is completed, the resin was filtered off, and the filtrate was slowly poured into previously cooled methyl tert-butyl ether (500 mL). the addition was conducted dropwise while stirring. The solution was settled in an ice bath for 2 hours. The supernatant was removed, and previously cooled methyl tert-butyl ether was added to centrifuge and wash for 5 times, each time 350 mL.

Thereafter, the material was collected, and dried under reduced pressure at room temperature to obtain 2.4 g crude peptide.

### Purification of Peptide:

The crude peptide was refined through multi-step purification: the first step: stationary phase: C18 (Kromsil: 10 μm-100À), linear gradient for the mobile phase 20-60% B (mobile phase A: 0.1% TFA, mobile phase B: acetonitrile), 40 minutes, ultraviolet (UV) detection at 214 nm; the second step: stationary phase: C18 (Kromsil: 10 μm-100À), linear gradient for the mobile phase 20-60% B (mobile phase A: 0.5% phosphoric acid, mobile phase B: acetonitrile), 40 minutes, ultraviolet (UV) detection at 214 nm. Finally, the refined peptide (97.9%) was obtained by lyophilization. MS: m/z 4329.07 (M+H)+.

The chemical name or chemical structure of Peptide 7 is

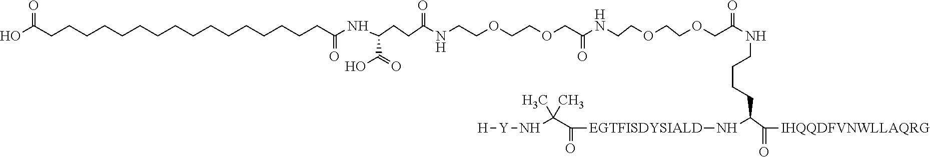

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R30]-hGIP(1-31).

### Example 3: Preparation of Long-Acting Conjugate of Sequence 8 (Peptide 8)

The following amino acids were used in the synthesis for extending the polypeptides in Example 3: Fmoc-L-Tyr(tBu)-OH; Fmoc-L-Aib-OH; Fmoc-LGlu(OtBu)-OH; Fmoc-L-Gly-OH; Fmoc-L-Thr(tBu)-OH; Fmoc-L-Phe-OH; Fmoc-L-Ile-OH; Fmoc-L-Ser(tBu)-OH; F-moc-L-Asp(OtBu)-OH; Fmoc-L-Tyr(tBu)-OH; Fmoc-L-Ser(tBu)-OH; Fmoc-L-Ile-OH; Fmoc-Ala-OH; Fmoc-L-Leu-OH; Fmoc-L-Asp(OtBu)-OH; Fmoc-L-Lys(Mtt)-OH; Fmoc-L-Leu-OH; Fmoc-L-His(Trt)-OH; Fmoc-L-Gln(Trt)-OH; Fmoc-L-Gln(Trt)-OH; Fmoc-L-Asp(OtBu)-OH; Fmoc-L-Phe-OH; Fmoc-L-Val-OH; Fmoc-L-Asn(Trt)-OH; Fmoc-L-Trp(Boc)-OH; Fmoc-L-Leu-OH; Fmoc-L-Leu-OH; Fmoc-Ala-OH; Fmoc-L-Gln(Trt)-OH; Fmoc-Arg(Pbf)-OH; and Fmoc-L-Gly-OH.

The synthesis method is the same as that described in Example 2. The obtained crude peptide is purified by RP-HPLC, and finally the refined peptide (98.2%) was obtained by lyophilization. MS: 4329.07 (M+H)+.

The chemical name or chemical structure of peptide 8 is

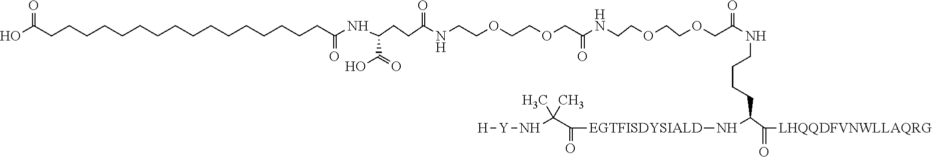

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, R30]-hGIP(1-31)

### Example 4: Preparation of Long-Acting Conjugate of Sequence 9 (Peptide 9)

The following amino acids were used in the synthesis for extending the polypeptides in Example 4: Fmoc-L-Tyr(tBu)-OH; Fmoc-L-Aib-OH; Fmoc-L-Glu(OtBu)-OH; Fmoc-L-Gly-OH; Fmoc-L-Thr(tBu)-OH; Fmoc-L-Phe-OH; Fmoc-L-Ile-OH; Fmoc-L-Ser(tBu)-OH; F-moc-L-Asp(OtBu)-OH; Fmoc-L-Tyr(tBu)-OH; Fmoc-L-Ser(tBu)-OH; Fmoc-L-Ile-OH; Fmoc-Ala-OH; Fmoc-L-Leu-OH; Fmoc-L-Asp(OtBu)-OH; Fmoc-L-Lys(Mtt)-OH; Fmoc-L-Ile-OH; Fmoc-L-His(Trt)-OH; Fmoc-L-Gln(Trt)-OH; Fmoc-L-Glu(OtBu)-OH; F-moc-L-Asp(OtBu)-OH; Fmoc-L-Phe-OH; Fmoc-L-Val-OH; Fmoc-L-Asn(Trt)-OH; Fmoc-L-Trp(Boc)-OH; Fmoc-L-Leu-OH; Fmoc-L-Leu-OH; Fmoc-Ala-OH; Fmoc-L-Gln(Trt)-OH; Fmoc-Arg(Pbf)-OH; and Fmoc-L-Gly-OH.

The synthesis method is the same as that described in Example 2. The obtained crude peptide is purified by RP-HPLC, and finally the refined peptide (98.2%) was obtained by lyophilization. MS: 4330.05 (M+H)+.

The chemical name or chemical structure of Peptide 9 is

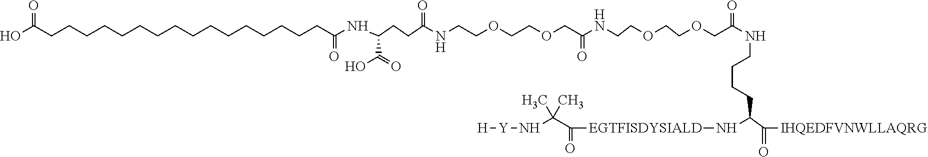

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, E20, R30]-hGIP(1-31).

### Example 5: Preparation of Long-Acting Conjugate of Sequence 10 (Peptide 10)

The following amino acids were used in the synthesis for extending the polypeptides in Example 5: Fmoc-L-Tyr(tBu)-OH; Fmoc-L-Aib-OH; Fmoc-L-Glu(OtBu)-OH; Fmoc-L-Gly-OH; Fmoc-L-Thr(tBu)-OH; Fmoc-L-Phe-OH; Fmoc-L-Ile-OH; Fmoc-L-Ser(tBu)-OH; F-moc-L-Asp(OtBu)-OH; Fmoc-L-Tyr(tBu)-OH; Fmoc-L-Ser(tBu)-OH; Fmoc-L-Ile-OH; Fmoc-Ala-OH; Fmoc-L-Leu-OH; Fmoc-L-Asp(OtBu)-OH; Fmoc-L-Lys(Mtt)-OH; Fmoc-L-Leu-OH; Fmoc-L-His(Trt)-OH; Fmoc-L-Gln(Trt)-OH; Fmoc-L-Glu(OtBu)-OH; Fmoc-L-Asp(OtBu)-OH; Fmoc-L-Phe-OH; Fmoc-L-Val-OH; Fmoc-L-Asn(Trt)-OH; Fmoc-L-Trp(Boc)-OH; Fmoc-L-Leu-OH; Fmoc-L-Leu-OH; Fmoc-Ala-OH; Fmoc-L-Gln(Trt)-OH; Fmoc-Arg(Pbf)-OH; and Fmoc-L-Gly-OH.

The synthesis method is the same as that described in Example 2. The obtained crude peptide is purified by RP-HPLC, and finally the refined peptide (97.5%) was obtained by lyophilization. MS: 4330.05 (M+H)+.

The chemical name or chemical structure of Peptide 10 is

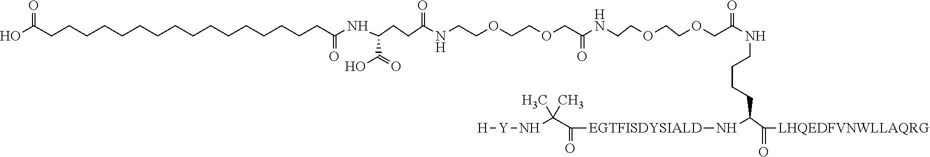

N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, E20, R30]-hGIP(1-31).

### Example 6: Preparation of Long-Acting Conjugate of Sequence 14 (Peptide 14)

The following amino acids were used in the synthesis for extending the polypeptides in Example 6: Fmoc-L-Tyr(tBu)-OH; Fmoc-L-Aib-OH; Fmoc-L-Glu(OtBu)-OH; Fmoc-L-Gly-OH; Fmoc-L-Thr(tBu)-OH; Fmoc-L-Phe-OH; Fmoc-L-Ile-OH; Fmoc-L-Ser(tBu)-OH; Fmoc-L-Asp(OtBu)-OH; Fmoc-L-Tyr(tBu)-OH; Fmoc-L-Ser(tBu)-OH; Fmoc-L-Il-OH; Fmoc-Ala-OH; Fmoc-L-Leu-OH; Fmoc-L-Asp(OtBu)-OH; Fmoc-Arg(Pbf)-OH; Fmoc-L-Ile-OH; Fmoc-L-His(Trt)-OH; Fmoc-L-Gln(Trt)-OH; Fmoc-L-Gln(Trt)-OH; Fmoc-L-Asp(OtBu)-OH; Fmoc-L-Phe-OH; Fmoc-L-Val-OH; Fmoc-L-Asn(Trt)-OH; Fmoc-L-Trp(Boc)-OH; Fmoc-L-Leu-OH; Fmoc-L-Leu-OH; Fmoc-Ala-OH; Fmoc-L-Gln(Trt)-OH; Fmoc-L-Lys(Mtt)-OH; and Fmoc-L-Gly-OH.

The synthesis method is the same as that described in Example 2. The obtained crude peptide is purified by RP-HPLC, and finally the refined peptide (95.8%) was obtained by lyophilization. MS: 4328.97 (M+H)+.

The chemical name or chemical structure of Peptide 14 is

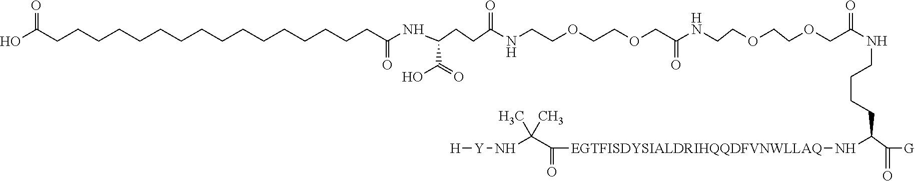

N-{ε-30}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R16]-hGIP(1-31).

The synthesis and preparation methods of the remaining peptides are carried out with reference to the technical solutions described in Examples 2 to 6.

### Example 7: Cell Activity Assay

GIP receptor (glucose-dependent insulinotropic peptide receptor, GIPR) belongs to G protein-coupled receptors, which is mainly coupled with Gs proteins. When the GIP receptor is activated, the activity of adenylyl cyclase (AC) is stimulated by the Gs proteins, thereby the intracellular cAMP level is increased. The HTRF CAMP assay is a competitive immunoassay for the detection of changes in intracellular cAMP. The detection principle is that fluorescence resonance energy transfer (FRET) can occur after Eu-labeled CAMP is bonded to ULight™ dye-labeled ULight™-anti-cAMP antibody, while unlabeled cAMP (such as cAMP produced by cells) and the Eu-labeled CAMP can competitively bond to the Anti-cAMP antibody, resulting in a decrease in the FRET signal. That is, the signal value is inversely related to the concentration of cAMP. The present invention utilizes a stably transduced cell line expressing GIPR HEK293, and after incubating it with tested compounds at different concentrations, the agonistic activity of the compounds at GIPR was evaluated by measuring cAMP level using an HTRF kit.

HEK cell line stably expressing GIPR was incubated in a medium DMEM containing 10% FBS (fetal bovine serum)+100 μg/mL Hygromycin B+10% FBS at a culture temperature of 37° C. and a carbon dioxide concentration of 5%. The old culture medium was removed, and the cell line was washed once with PBS. Then, 1 mL TrypLE™ Express solution was added. Incubation was conducted at 37ºC for about 2 minutes. When the cells detach from the bottom of the dish, about 5 mL of complete culture medium preheated at 37° ° C. was added. The cell suspension was gently blown with a pipette, thereby separating the aggregated cells. The cell suspension was transferred into a sterile centrifuge tube and centrifuged at 1000 rpm for 5 minutes. The cells were collected for further test or subculture.

The agonistic activity of the compounds on the receptor was determined using the HTRF kit (purchased from Perkin Elmer). All reagents in the kit were allowed to equilibrate to room temperature before use. A 1× Stimulation Buffer was formulated according to the instruction in the kit for use. Glucagon, a compound exhibits positive in the GCGR binding assay, was gradiently diluted with DMSO, and then 100-fold diluted with 1× Stimulation Buffer to obtain a 10× working solution. GIP, a compound exhibits positive in the GIPR binding assay, was gradiently diluted with 1% DMSO Buffer to 10×. GIPR cells were digested with trypsin, centrifuged to remove the culture medium. The cells were resuspended in the 1× Stimulation Buffer. After being counted, 9 μL cell dilutions were inoculated into a 384-well plate at 1000 cells/well. 1 μL the 10× compound diluted in step 2 was taken and added to the corresponding test well, wherein 1 μL the 10× initial concentration positive compound was added to the PC hole (Min value), and 1 μL the 10×DMSO buffer (1%) was added to the VC hole (Max value). The plate was centrifuged and incubated at 37ºC for 30 minutes. Eu-cAMP was diluted with Detection buffer to a working concentration, and the diluent was added into the corresponding test well at 5 μL/well. ULight-anti-Camp was diluted with Detection buffer to a working concentration, and the diluent was added into the corresponding test well at 5 μL/well. After being centrifuged, the plate was incubated at room temperature for 1 hour. After the incubation is completed, the measurements were obtained using a Biotek multifunctional microplate reader at 665 nm and 620 nm. The Ratio (665/620) was plotted against the concentration of the compound. The nonlinear regression method of GraphPad Prism software was used for curve fitting and EC50 calculation.

TABLE 1
Cell Agonistic Activity of Candidate Sequences

| GIP Analog Sequence | hGIPR | hGLP-1R |
|---|---|---|
| (naked peptide) No. | EC50(pM) | EC50(pM) |
| hGIP (1-42) | 5.11 | >100 |
| hGLP-1 (7-37) | >100 | 7.01 |
| Sequence 1 (Seq ID. 1) | 2.38 | >100 |
| Sequence 2 (Seq ID. 2) | 10.69 | >100 |
| Sequence 3 (Seq ID. 3) | 3.41 | >100 |
| Sequence 4 (Seq ID. 4) | 2.87 | >100 |
| Sequence 5 (Seq ID. 5) | 6.12 | >100 |
| Sequence 6 (Seq ID. 6) | 7.93 | >100 |
| Sequence 7 (Seq ID. 7) | 6.04 | >100 |
| Sequence 8 (Seq ID. 8) | 11.66 | >100 |
| Sequence 9 (Seq ID. 9) | 16.42 | >100 |
| Sequence 10 (Seq ID. 10) | 1.63 | >100 |
| Sequence 11 (Seq ID. 11) | 3.44 | >100 |
| Sequence 12 (Seq ID. 12) | 0.81 | >100 |
| Sequence 13 (Seq ID. 13) | 36.2 | >100 |
| Sequence 14 (Seq ID. 14) | 18.4 | >100 |
| Sequence 15 (Seq ID. 15) | 27.24 | >100 |
| Sequence 16 (Seq ID. 16) | 25.5 | >100 |
| Sequence 17 (Seq ID. 17) | 58.21 | >100 |
| Sequence 18 (Seq ID. 18) | 16.88 | >100 |

TABLE 2
Cell Agonistic Activity of Candidate Compounds

| Long-acting Conjugate | hGIPR | hGLP-1R |
|---|---|---|
| of GIP Analog No. | EC50(pM) | EC50(pM) |
| Peptide 5 | 26.98 | >100 |
| Peptide 6 | 32.16 | >100 |
| Peptide 7 | 6.66 | >100 |
| Peptide 8 | 4.26 | >100 |
| Peptide 9 | 7.24 | >100 |
| Peptide 10 | 6.82 | >100 |
| Peptide 11 | 3.50 | >100 |
| Peptide 12 | 5.14 | >100 |
| Peptide 13 | 5.61 | >100 |
| Peptide 14 | 3.5 | >100 |
| Peptide 15 | 9.14 | >100 |
| Peptide 16 | 3.77 | >100 |
| Peptide 17 | 25.68 | >100 |
| Peptide 18 | 21.09 | >100 |

Sequences 1-18 are the naked peptide forms of GIP analogs. Peptides 5-18 are long-acting conjugates of GIP analogs. The amino acid sequences of the GIP analogs correspond to Seq ID. 5-18.

The present invention restructures the sequence of naturally occurring GIP and modifies it with long-lasting conjugating components, resulting in GIP analogs and long-lasting conjugates thereof which have improved GIP receptor agonist activity as compared with the naturally occurring GIP, and selective activation of the GIP receptors was achieved.

### Example 8: IPGTT (Intraperitoneal Glucose Tolerance Test) to Investigate the Effect on Blood Glucose Level after Administrating Long-Acting Conjugates of GIP Analogs

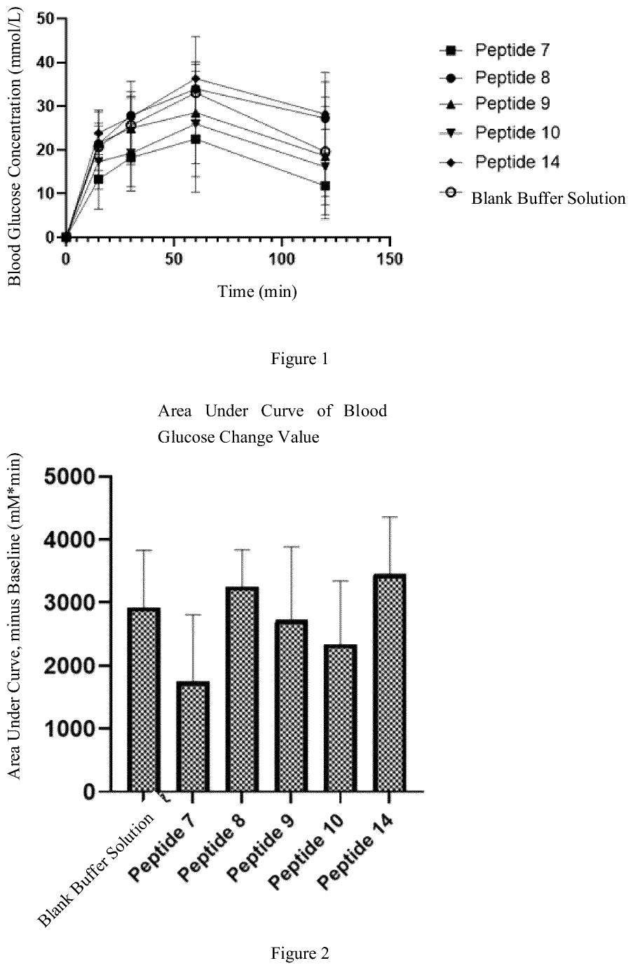

On the day of the glucose tolerance test, db/db male mice (8-9 weeks) were injected with a long-acting conjugate of GIP analog (modified by fatty acid chains): peptide 7, 8, 9, 10, 14 (see Examples 2, 3, 4, 5, and 6 for details as for the methods of preparation and chemical structures). Synchronously, the animal was fasted for 4 hours. Animals were allowed to drink water but not to eat. Tail blood glucose level was measured. The mice were intraperitoneally (i. p.) injected with glucose at a load of 2 g/kg (200 mg/mL glucose solution, dose volume 10 mL/kg) (t=0). Tail blood glucose levels were measured at 0, 15, 30, 60, and 120 mins after intraperitoneal injection of glucose. The values of blood glucose changes and the area under the curve after injection of each compound are shown in FIGS. 1 and 2.

It may be seen from the FIGS. 1 and 2 that the blood glucose tolerance caused by the use of the long-acting conjugates of GIP analogs of the present invention (Peptide 7, 9, and 10) were significantly improved, and Peptide 7 exhibits a particularly significant effect.

### Example 9: Male Type 2 Diabetic Mice Modeled with STZ were Continuously Administered with a Long-Acting Conjugate of GIP Analog in Combination with a GLP-1 Analog for 7 Days, and then Measured for the Fasting Blood Glucose. The Effect on the Blood Glucose was Evaluated

The GLP-1 analog used in this example is Semaglutide, which may be prepared as described in Example 4 of WO2006/097537 and is also known as N^{6,26}-{18-[N-(17-carboxylheptadecanoyl)-L-γ-glutamyl]-10-oxo-3,6,12,15-tetraoxa-9,18-diazaoctadecanoyl}-[8-(2-amino-2-propionic acid), 34-L-arginine] human glucagon-like peptide 1 (7-37), see WHO Drug Information Vol. 24, No. 1, 2010.

The long-acting conjugate of GIP analog used in this example is Peptide 7. The preparation method and chemical structure thereof in details may be seen in Example 2.

Male mice were allowed to adapt to new environment for 1 week before treatment. The mice were divided into several groups (n=6-8/group). The individual was screened and divided into groups based on body weight and fasting blood glucose. Grouping was done by screening in a balanced manner, so as to exclude individuals that were too large or too small. A blank buffer or a test compound was subcutaneously administrated to the animals once a day. The individuals in the body weight group were weighted daily before administration, while the individuals in the fasting blood glucose group (fasting for 4 hours) were measured for their blood glucose 7 days after administration.

TABLE 3
Hypoglycemic Efficacy of Long-acting Conjugates
of GIP analogs in Combination with GLP-1 Analogs

| | | | | Decrease |
|---|---|---|---|---|
| | Dosage | Fasting Blood Glucose | | of Blood |
| | (nmol/ | Value (mmol/L) | | Glucose (%) |
| Compound | kg) | 0 day | 7 days | 7 days |
| Blank Buffer | / | 21.86 ± 1.81 | 20.78 ± 1.39 | 4.94 ± 12.84 |
| GLP-1 Analog | 14.4 | 26.96 ± 1.60 | 13.66 ± 4.94 | 49.30 ± 17.03 |
| GLP-1 Analog | 2.4 | 27.17 ± 2.01 | 19.82 ± 3.79 | 27.10 ± 13.55 |
| Peptide 7 | 45 | / | / | / |
| GLP-1 | 2.4 + 45 | 25.84 ± 2.06 | 15.42 ± 3.08 | 40.30 ± 12.48 |
| Analog + | | | | |
| Peptide 7 | | | | |

It may be seen from Table 3 that single drug treatment with GLP-1 analogs resulted in a decrease in blood glucose values, and the decrease was in an obvious dosage-dependent manner. The combined treatment of GLP-1 receptor agonist (2.4 nmol/kg) and Peptide 7 (45 nmol/kg) enhanced the effect of lowering blood glucose. The combined treatment was significantly better than the improvement achieved by single drug treatment, and was better than cumulative effect.

### Example 10: Weight Loss Efficacy of Long-Acting Conjugates of GIP Analogs in Combination with GLP-1 Analogs

The GLP-1 analog used in this example is the same as Example 9, namely Semaglutide. Eight or more C57BL/6J male mice at an age of 16 to 18 weeks and fifty-six or more DIO male mice with obesity induced by high-fat feed at an age of 16 to 18 weeks were selected. After entering the animal facility, all mice were adaptively fed for 3 days or more. They were required to adapt for 3 days before grouping. All mice were measured for body weight, body fat ratio and fasting blood glucose. Fifty-six DIO mice were randomly grouped primarily based on body weight and fasting blood glucose and secondarily based on body fat ratio. 8 C57BL/6J mice of close weekly age were assigned into the healthy control group. Each mouse in all groups was housed in a separate cage for 2-3 days to adapt to environment before grouping. During the period, the mice were adaptively caught and administrated with blank solvent daily. The day of grouping was Day 0, and the administration was continued for 28 days. The Vehicle group was administrated with blank solvent (propylene glycol: 14.0 mg/mL and disodium hydrogen phosphate: 1.132 mg/mL, pH 7.4); s.c. means subcutaneous injection; the frequency of administration QD means once a day. The specific dosing schedule was shown in the following Table.

TABLE 4
Dosing Schedule-1

| | Number of | | | Dosage | Administration | |
|---|---|---|---|---|---|---|
| Group | Animals | Strains | Treatment | (nmol/kg) | Route | Frequency |
| G1 | 8 | C57BL/6J | Vehicle | — | s.c. | QD |
| G2 | 8 | DIO | Vehicle | — | s.c. | QD |
| G3 | 8 | DIO | GLP-1 Analog | 2 | s.c. | QD |
| G4 | 8 | DIO | GLP-1 Analog + Peptide 7 | 2 + 90 | s.c. | QD |
| G5 | 8 | DIO | GLP-1 Analog + Peptide 8 | 2 + 90 | s.c. | QD |
| G6 | 8 | DIO | GLP-1 Analog + Peptide 9 | 2 + 90 | s.c. | QD |
| G7 | 8 | DIO | GLP-1 Analog + Peptide 10 | 2 + 90 | s.c. | QD |
| G8 | 8 | DIO | Peptide 7 | 90 | s.c. | QD |

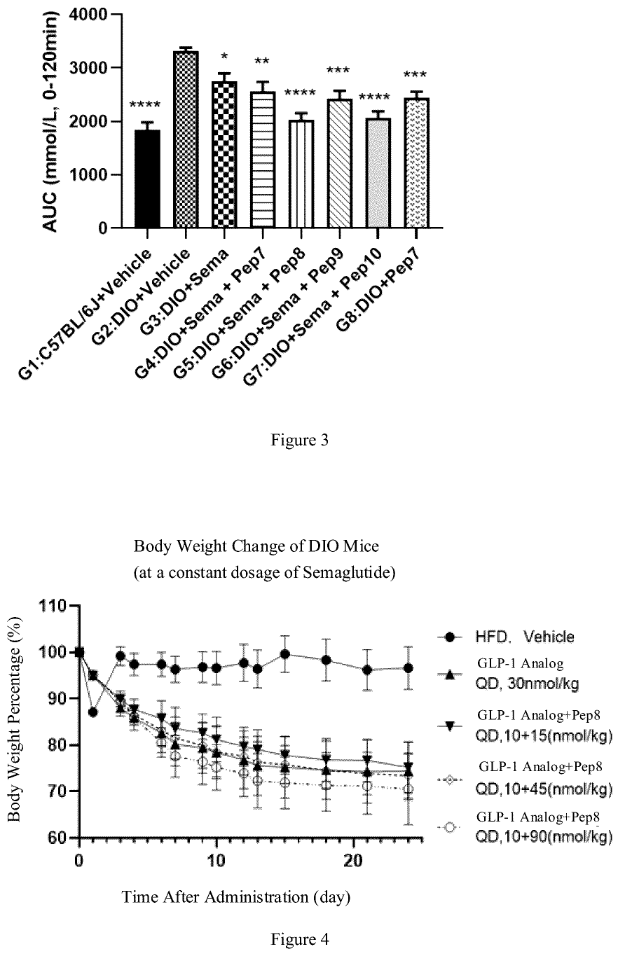

The body weight of each mouse was recorded every day according to the frequency of administration. Postprandial blood glucose was measured every 3 days, fasting blood glucose was measured every 7 days, and IPGTT (Intraperitoneal Glucose Tolerance Tests) was conducted once before the end point. It can be seen from the data of body weight in Table 5 that the GIP analogs have limited efficacy when administrated alone. However, the combination use of GIP analogs can significantly enhance the weight loss efficacy of GLP-1 analogs, and the effect thereof is better than a cumulative effect. It may be seen from the AUC of blood glucose change value in IPGTT conducted before the end point as shown in FIG. 3 that the combination use of a GLP-1 receptor agonist and a GIP analog improves the effect of reducing blood glucose value (as compared with the G2&G3 groups). Among the GIP analogs, Peptide 8 exhibits the best effect in the combination use.

TABLE 5
Body Weight Change after 28 days of Administration

| Group | Weight (g) | Weight Change |
|---|---|---|
| G1 | 30.96 ± 1.02 | +2.05% |
| G2 | 44.0 ± 2.4 | +7.65% |
| G3 | 40.2 ± 1.5 | −3.2% |
| G4 | 33.3 ± 0.8 | −22.4% |
| G5 | 32.9 ± 0.9 | −23.4% |
| G6 | 34.7 ± 1.3 | −14.4% |
| G7 | 34.5 ± 0.9 | −17.1% |
| G8 | 44.6 ± 1.0 | +3.2% |

### Example 11: Male DIO Mice were Continuously Administered with a Long-Acting Conjugate of GIP Analog in Combination with a GLP-1 Analog at Different Administration Ratios for 25 Days, and then Measured for the Body Weight. The Effects on Food Intake, Total Liver Cholesterol (TC) Level and Total Triglyceride (TG) Level were Evaluated

The GLP-1 analog used in this example is the same as that in Example 8, i.e., Semaglutide. The long-acting conjugate of GIP analog used in this example is Peptide 8. The preparation method and chemical structure thereof in details may be seen in Example 3.

Sixty-four high-fat diet-induced obese (DIO) mice were weighed. When the average weight of the mice is about 40-45 g, they are randomly assigned to Groups G2-G11 based on the body weight of the mice, with 8 mice in each group. Eight C57BL/6 mice fed with normal diet were assigned to Group G1. Each mouse in all groups was housed in a separate cage for 2-3 days to adapt to environment before grouping. During this period, the mice were adaptively caught and administrated with blank solvent daily. The day of grouping was Day 0. The specific dosing schedule was shown in the following Table 6. The Vehicle group was administrated with blank solvent (propylene glycol: 14.0 mg/mL and disodium hydrogen phosphate: 1.132 mg/mL) at pH 7.4; s.c. means subcutaneous injection; the frequency of administration QD means once a day. Such expressions may be applied to the following Examples.

TABLE 6
Dosing Schedule-2

| | | Number of | Dosage | Administration | Administration |
|---|---|---|---|---|---|
| Group | Test Material | Animals | (nmol/kg) | Route | Frequency |
| G1 | Vehicle | 8 | — | s.c. | QD |
| G2 | Vehicle | 8 | — | s.c. | QD |
| G3 | GLP-1 Analog | 8 | 30 | s.c. | QD |
| G4 | GLP-1 Analog + Peptide 8 | 8 | 5 + 90 | s.c. | QD |
| G5 | GLP-1 Analog + Peptide 8 | 8 | 10 + 90 | s.c. | QD |
| G6 | GLP-1 Analog + Peptide 8 | 8 | 30 + 90 | s.c. | QD |
| G7 | GLP-1 Analog + Peptide 8 | 8 | 10 + 45 | s.c. | QD |
| G8 | GLP-1 Analog + Peptide 8 | 8 | 10 + 15 | s.c. | QD |

The animals were weighed before grouping (i.e. before the first administration) and every 3 days after grouping. At the end of the experiment (Day 25), the mice were fasted for 4 hours. The livers of all mice in Groups G1-G8 were collected and weighed. About 20 mg of the livers were homogenized. The homogenate was taken to measure total cholesterol (TC) and triglyceride glycerol (TG).

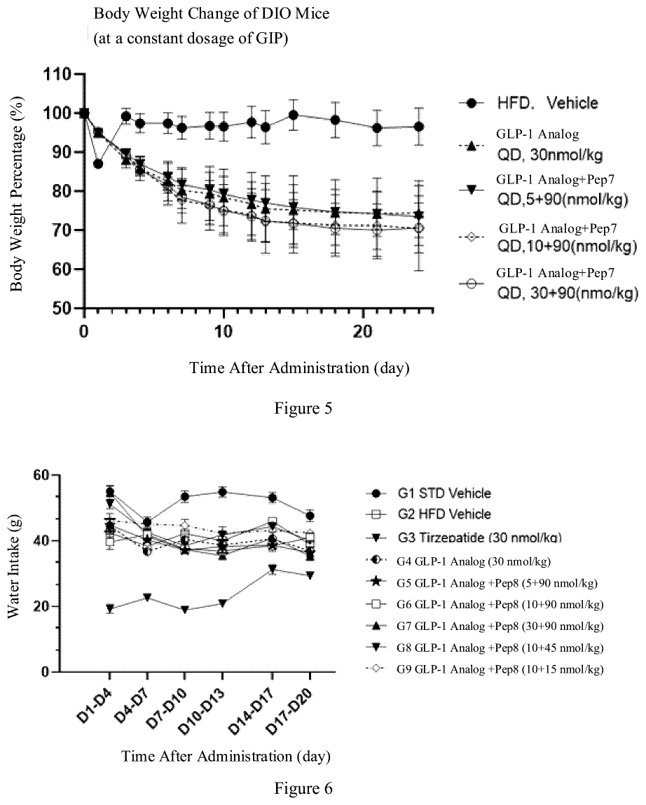

The test results were shown in Table 7, Table 8, FIG. 4 and FIG. 5. On Day 25, as compared with the G1 blank solvent control, the body weight of the G2 model group was significantly increased. As compared with the G2 model group, the body weight of the mice in other treatment groups was significantly reduced. The degree of the reduction exceeds that of the G2 model group by more than 20%. At a GLP-1 analog dosage of 10 nmol/kg, the weight loss efficacy produced after being administrated in combination with various dosages of Peptide 8 was equivalent to or superior to that produced at a GLP-1 dosage of 30 nmol/kg alone. At a Peptide 8 dosage of 90 nmol/kg and a low GLP-1 dosage (5 nmol/kg) to medium-high GLP-1 dosage (10, 30 nmol/kg), the efficacy of combination administration was equivalent to or superior to that produced at a GLP-1 dosage of 30 nmol/kg alone. By means of dosage combination, it was found that GIP analogs can enhance the therapeutic effect of GLP-1 analogs, and such combination use exhibits a synergistic effect. In addition to the efficacy in weight loss, the combination use of GIP analogs and GLP-1 analogs also has the effect of enhancing the improvement of TG/TC metabolic indicators.

TABLE 7
Weight Loss Efficacy of GLP-1 Analog at a Constant Dosage after Being
Administrated in Combination with Various Dosages of Peptide 8

| | | | | Ratio of Body Weight after |
|---|---|---|---|---|
| | | Dosage | Body | Administration to Body Weight |
| Group | Test Material | (nmol/kg) | Weight (g) | Before Administration (%) |
| G2 | Vehicle | — | 39.3 ± 0.9 | 93.4 ± 1.5 |
| G3 | GLP-1 Analog | 30 | 31.1 ± 0.7 **** | 74.4 ± 1.6 **** |
| G8 | GLP-1 Analog + Peptide 8 | 10 + 15 | 31.5 ± 0.4 **** | 75.2 ± 1.5 **** |
| G7 | GLP-1 Analog + Peptide 8 | 10 + 45 | 30.8 ± 0.4 **** | 73.4 ± 1.2 **** |
| G5 | GLP-1 Analog + Peptide 8 | 10 + 90 | 29.6 ± 0.8 **** | 70.5 ± 1.9 **** |

The body weights of G1 blank solvent control group, G3-G10 treatment groups, and G2 model group were measured at each time point. Statistical analysis was conducted on the body weight change percentage, (One-way ANOVA),
* p < 0.5,
** p < 0.01,
*** p < 0.001,
**** p < 0.0001.

TABLE 8
Weight Loss Efficacy of Peptide 8 at a Constant Dosage after Being
Administrated in Combination with Various Dosages of GLP-1 Analog

| | | | | Ratio of Body Weight after |
|---|---|---|---|---|
| | | Dosage | Body | Administration to Body Weight |
| Group | Test Material | (nmol/kg) | Weight (g) | Before Administration (%) |
| G2 | Vehicle | — | 39.3 ± 0.9 | 93.4 ± 1.5 |
| G3 | GLP-1 Analog | 30 | 31.1 ± 0.7 *** | 74.4 ± 1.6 **** |
| G4 | GLP-1 Analog + Peptide 8 | 5 + 90 | 30.9 ± 0.5 *** | 73.5 ± 1.0 **** |
| G5 | GLP-1 Analog + Peptide 8 | 10 + 90 | 29.6 ± 0.8 *** | 70.5 ± 1.9 **** |
| G6 | GLP-1 Analog + Peptide 8 | 30 + 90 | 29.6 ± 0.4 *** | 70.6 ± 1.1 **** |

The body weights of G1 blank solvent control group, G3-G10 treatment groups, and G2 model group were measured at each time point. Statistical analysis was conducted on the body weight change percentage, (One-way ANOVA),
* p < 0.5,
** p < 0.01,
*** p < 0.001,
**** p < 0.0001.

TABLE 9
Effect of Test Material on Test Endpoints TG and TC in DIO mice

| | | Dosage | TC | TG |
|---|---|---|---|---|
| Group | Test Material | (nmol/kg) | (mM) | (mM) |
| G1 | Vehicle | — | 2.88 ± 0.03 **** | 0.73 ± 0.04 **** |
| G2 | Vehicle | — | 5.90 ± 0.13 | 1.10 ± 0.03 |
| G3 | GLP-1 Analog | 30 | 4.89 ± 0.12 **** | 0.91 ± 0.03 * |
| G4 | GLP-1 Analog + Peptide 8 | 5 + 90 | 4.26 ± 0.11 **** | 1.07 ± 0.04 |
| G5 | GLP-1 Analog + Peptide 8 | 10 + 90 | 3.69 ± 0.28 *** * | 0.83 ± 0.03 |
| G6 | GLP-1 Analog + Peptide 8 | 30 + 90 | 4.20 ± 0.10 **** | 1.06 ± 0.04 |
| G7 | GLP-1 Analog + Peptide 8 | 10 + 45 | 3.98 ± 0.09 ** * * | 1.06 ± 0.04 * ** |
| G8 | GLP-1 Analog + Peptide 8 | 10 + 15 | 4.43 ± 0.08 **** | 1.09 ± 0.06 |

The body weights of G1 blank solvent control group, G3-G10 treatment groups, and G2 model group were measured at each time point. Statistical analysis was conducted on the change of TC/TG, (One-way ANOVA),
* p < 0.5,
** p < 0.01,
*** p < 0.001,
**** p < 0.0001.

### Example 12: Investigation of Efficacy of Long-Acting Conjugate of GIP Analog at High Dosage

The GLP-1 analog used in this example is the same as that in Example 8, i.e., Semaglutide.

The long-acting conjugates of GIP analogs used in this example is Peptide 7 and Peptide 8. The preparation method and chemical structure thereof in details may be seen in Example 2 and Example 3.

72 diet-induced obese (DIO) mice were weighed. (Screening and Grouping) When the average weight of the mice is about 40-45 g, they are randomly assigned to Groups G2-G11 based on the body weights of the mice, with 8 mice in each group. Eight C57BL/6 mice fed with normal diet were assigned to Group G1. Each mouse in all groups was housed in a separate cage for 2-3 days to adapt to environment before grouping. During this period, the mice were adaptively caught and administrated with blank solvent (propylene glycol: 14.0 mg/mL and disodium hydrogen phosphate: 1.132 mg/mL, pH 7.4) daily. The day of grouping was Day 0. The specific dosing schedule was shown in the following Table 10.

By varying the ratio of GLP-1 analog (Semaglutide) to the long-acting conjugate of GIP analog (Peptide 7 or Peptide 8), at a high dosage of GLP-1 (30 nmol/kg), when Peptide7 or Peptide 8 was administrated in combination with GLP-1 at a ratio such as 1:1, 1:3, 1:9 can still further expand the weight loss efficacy. The results are shown in Table 11.

TABLE 10
Dosing Schedule-3

| | Number | | | | |
|---|---|---|---|---|---|
| | of | dosage | Administration | Administration | Administration |
| Test Material | Animals | (nmol/kg) | Route | Frequency | Times |
| Vehicle | 8 | — | s.c. | QD | 21 |
| Vehicle | 8 | — | s.c. | QD | 21 |
| GLP-1 Analog | 8 | 30 | s.c. | QD | 21 |
| GLP-1 Analog + Peptide 7 | 8 | 30 + 30 | s.c. | QD | 21 |
| GLP-1 Analog + Peptide 7 | 8 | 30 + 90 | s.c. | QD | 21 |
| GLP-1 Analog + Peptide 7 | 8 | 30 + 270 | s.c. | QD | 21 |
| GLP-1 Analog + Peptide 8 | 8 | 30 + 30 | s.c. | QD | 21 |
| GLP-1 Analog + Peptide 8 | 8 | 30 + 90 | s.c. | QD | 21 |
| GLP-1 Analog + Peptide 8 | 8 | 30 + 150 | s.c. | QD | 21 |
| GLP-1 Analog + Peptide 8 | 8 | 30 + 270 | s.c. | QD | 21 |

TABLE 11
Effect of Test Material on Body Weight of DIO Mice on Day 21 (21 days after administration)

| | | | | Body Weight | Body Weight |
|---|---|---|---|---|---|
| | | Dosage | Body | Percentage at | Reduction Percentage |
| Group | Test Material | (nmol/kg) | Weight (g) | Day 21 (%) | at Day 21 (% ) |
| G1 | Vehicle 2 | — | 29.1 ± 0.3 **** | 96.9 ± 0.6 | 3.7 ± 0.8 |
| G2 | Vehicle 2 | — | 41.4 ± 0.9 | 93.3 ± 0.9 | 8.2 ± 1.0 |
| G3 | GLP-1 Analog | 30 | 33.1 ± 0.7 **** | 74.6 ± 0.9 **** | 24.7 ± 1.3 **** |
| G4 | GLP- 1 Analog + Peptide 7 | 30 + 30 | 31.1 ± 0.6 **** | 69.9 ± 1.5 **** | 29.1 ± 1.6 **** |
| G5 | GLP-1 Analog + Peptide 7 | 30 + 90 | 29.8 ± 1.0 **** | 67.0 ± 1.8 **** | 29.0 ± 1.7 **** |
| G6 | GLP-1 Analog + Peptide 7 | 30 + 270 | 29.2 ± 0.4 **** | 65.5 ± 1.2 **** | 32.6 ± 1.1 **** |
| G7 | GLP-1 Analog + Peptide 8 | 30 + 30 | 31.6 ± 0.5 **** | 71.1 ± 1.1 **** | 28.0 ± 1.5 **** |
| G8 | GLP-1 Analog + Peptide 8 | 30 + 90 | 30.6 ± 0.5 **** | 69.1 ± 1.9 **** | 29.8 ± 1.8 **** |
| G9 | GLP-1 Analog + Peptide 8 | 30 + 150 | 30.6 ± 0.8 **** | 68.8 ± 1.1 **** | 29.2 ± 1.4 **** |
| G10 | GLP-1 Analog + Peptide 8 | 30 + 270 | 29.2 ± 0.5 **** | 66.2 ± 1.8 **** | 31.0 ± 1.6 **** |

The body weights of G1 blank solvent control group, G3-G10 treatment groups, and G2 model group were measured at each time point. Statistical analysis was conducted on the body weight change percentage, (One-way ANOVA),
* p < 0.5,
** p < 0.01,
*** p < 0.001,
**** p < 0.0001.

### Example 13: Effect of Long-Acting Conjugate of GIP Analog on Water Intake

Tirzepatide used in this example is a GIP and GLP-1 co-agonist compound, which has the peptide sequence of: H-Tyr¹-Aib²-Glu³-Gly⁴-Thr⁵-Phe⁶-Thr⁷-Ser⁸-Asp⁹-Tyr¹⁰-Ser¹¹-Ile¹². Aib¹³-Leu¹⁴-Asp¹⁵-Lys¹⁶-Ile¹⁷-Ala¹⁸-Gln¹⁹-Lys²⁰(AEEA-AEEA-γ-Glu-eicosanedioicacid)-Ala²¹_Phe²²-Val²³-Gln²⁴-Trp²⁵-Leu²⁶-Ile²⁷-Ala²⁸-Gly²⁹-Gly³⁰-Pro³¹-Ser³²-Ser³³-Gly³⁴-Ala³⁵-Pro³⁶-Pro³⁷-Pro³⁸-Ser³⁹-NH₂.

The compound may be prepared as described in Example 1 of WO 2016111971A1.

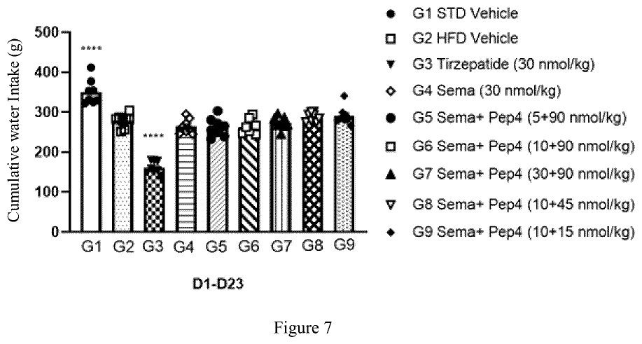

Based on the assay in Example 11, the cumulative water intake of the mice was also statistically calculated and compared with the water intake of the mice in the Tirzepatide treatment group. From the water intake data in Table 12, FIG. 6 and FIG. 7, it may be found that the water intake of the mice in the Tirzepatide treatment group was significantly inhibited, however the introduction of the long-acting conjugate of GIP analog in the test groups G5-G9 increases no additional adverse responses to the mice while improving the efficacy of GLP-1 analogs.

TABLE 12
Water Intake Data

| | | Dosage | Body | Cumulative Water | Average Water |
|---|---|---|---|---|---|
| Group | Test Material | (nmol/kg) | Weight (g) | Intake (g) | Intake (g) |
| G1 | Vehicle | — | 28.8 ± 0.5 **** | 350.5 ± 11.0 **** | 18.0 ± 0.6 **** |
| G2 | Vehicle | — | 39.3 ± 0.9 | 278.1 ± 6.0 | 14.3 ± 0.3 |
| G3 | Tirzepatide | 30 | 29.7 ± 1.2 *** * | 161.4 ± 5.5 **** | 8.3 ± 0.3 **** |
| G4 | GLP-1 Analogs | 30 | 31.1 ± 0.7 **** | 264.6 ± 6.5 | 13.6 ± 0.3 |
| G5 | GLP-1 Analog + Peptide 8 | 5 + 90 | 30.9 ± 0.5 **** | 262.3 ± 8.1 | 13.4 ± 0.4 |
| G6 | GLP-1 Analog + Peptide 8 | 10 + 90 | 29.6 ± 0.8 **** | 264.1 ± 6.3 | 13.5 ± 0.3 |
| G7 | GLP-1 Analog + Peptide 8 | 30 + 90 | 29.6 ± 0.4 **** | 273.2 ± 5.6 | 14.0 ± 0.3 |
| G8 | GLP-1 Analog + Peptide 8 | 10 + 45 | 30.8 ± 0.4 **** | 287.7 ± 3.9 | 14.8 ± 0.2 |
| G9 | GLP-1 Analog + Peptide 8 | 10 + 15 | 31.5 ± 0.4 **** | 290.9 ± 7.9 | 14.9 ± 0.4 |

The water intakes of G1 blank solvent control group, G3-G10: treatment groups, and G2 model group were measured at each time point. Statistical analysis was conducted on the water intake, (One-way ANOVA),
* p < 0.5,
** p < 0.01,
*** p < 0.001,
**** p < 0.0001.

What is claimed is:

1. A glucose-dependent insulinotropic polypeptide (GIP) analog, comprising a GIP-like polypeptide segment, wherein the GIP-like polypeptide segment is:
(a)
YX₂EGTFISDYSIX₁₃X₁₄DX₁₆X₁₇X₁₈QX₂₀X₂₁FVX₂₄WLLAQX₃₀X₃₁;
wherein X₂ is selected from A, D-Ala or Aib, X₁₃ is selected from A or Aib, X₁₄ is selected from M or L, X₁₆ is selected from E, K or R, X₁₇ is selected from I or L, X₁₈ is selected from H or R, X₂₀ is selected from K, E or Q, X₂₁ is selected from D or E, X₂₄ is selected from K or N, X₃₀ is selected from K or R, and X₃₁ is G or is absent; or
(b)
YX₂EGTFISDYSIX₁₃X₁₄DX₁₆X₁₇X₁₈QX₂₀X₂₁FVX₂₄WLLAQX₃₀X₃₁Y₁,
Y₁ may be selected from GPSSGAPPPS, KPSSGAPPPS, GPSSGAPPS, PSSGAPPPS, or PSSGAPPS; or
(c) an amino acid sequence with a sequence identity of 90% or above to (a) or (b), or a polypeptide segment having the function of the polypeptide segment as defined in (a) or (b).

2. The GIP analog according to claim 1, comprising an amino acid sequence selected from SEQ ID NOs: 1 to 18.

3. A long-acting conjugate of GIP analog, characterized in that it is consisted of the GIP analog according to claim 1 and a long-acting conjugating component.

4. The long-acting conjugate of GIP analog according to claim 3, characterized in that the long-acting conjugating component is connected to the GIP analog; and the long-acting conjugating component is selected from the group consisting of fatty acid side chains, polymers, cholesterols, albumins and segments thereof, albumin-binding substances, polymers of repeating units having specific amino acid sequences, antibodies, antibody segments, FcRn-binding substances, in vivo connective tissues, nucleotides, fibronectin, transferrin, saccharides, heparin, and elastin.

5. The long-acting conjugate of GIP analog according to claim 4, characterized in that the fatty acid side chain is selected from C8 to C26 fatty acids, the fatty acid side chain is a linear or branched chain, and the fatty acid side chain is a monocarboxylic acid and/or a dicarboxylic acid.

6. The long-acting conjugate of GIP analog according to claim 5, characterized in that the fatty acid side chain is selected from HOOC(CH₂)₁₄CO—, HOOC(CH₂)₁₅CO—, HOOC(CH₂)₁₆CO—, HOOC(CH₂)₁₇CO—, HOOC(CH₂)₁₈CO—, HOOC(CH₂)₁₉CO—, HOOC(CH₂)₂₀CO—, HOOC(CH₂)₂₁CO— or HOOC(CH₂)₂₂CO—.

7. The long-acting conjugate of GIP analog according to claim 6, characterized in that it is selected from the group consisting of: 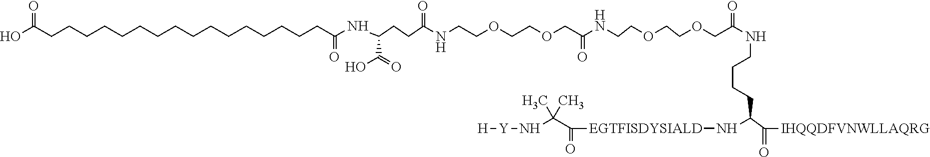
N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R30]-hGIP(1-31); 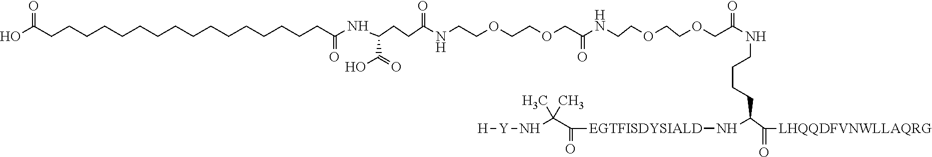
N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, R30]-hGIP(1-31); 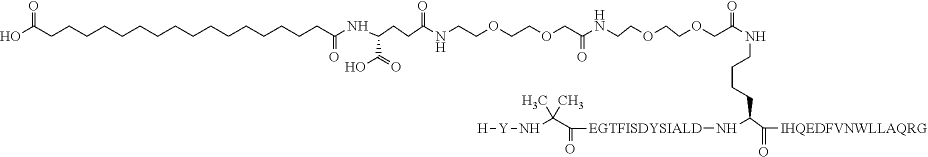
N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, E20, R30]-hGIP(1-31); 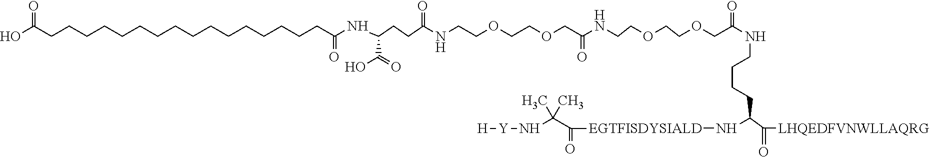
N-{ε-16}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, L17, E20, R30]-hGIP(1-31); and 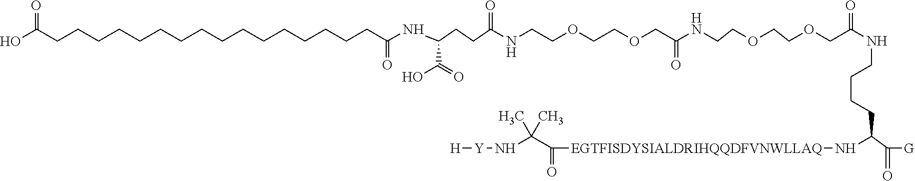
N-{ε-30}-18-{[(23R)-23-carboxyl-2,11,20-trioxo-10,19-diaza-4,7,13,16-tetraoxatricosyl-23-yl]amino}-18-oxooctadecanoic acid acetyl-[Aib2, L14, R16]-hGIP(1-31).

8. The GIP analog according to claim 1, characterized in that it has agonistic activity at GIP receptors, but has no agonistic activity at GLP-1 receptors.

9. A pharmaceutical composition, comprising the GIP analog according to claim 1, and at least one pharmaceutically acceptable excipient.

10. The pharmaceutical composition according to claim 9, characterized in that the composition is formulated into a liquid formulation suitable for injection or infusion, or an oral solid formulation.

11. The pharmaceutical composition according to claim 10, characterized in that the composition is formulated into a formulation which slowly releases the GIP analogue or the long-acting conjugate thereof in the composition.

12. The pharmaceutical composition according to claim 9, further comprising a GLP-1 receptor agonist, a glucagon receptor agonist, and a GLP-1/glucagon receptor coagonist.

13. A method for treating or preventing a disease selected from weight management, obesity and obesity-related diseases, all types of diabetes such as type 2 diabetes, and diabetes-related diseases, comprising administrating to a subject in need thereof, an effective amount of the GIP analog according to claim 1.

14. The long-acting conjugate of GIP analog according to claim 3, characterized in that it has agonistic activity at GIP receptors, but has no agonistic activity at GLP-1 receptors.

15. A pharmaceutical composition, comprising the long-acting conjugate of GIP analog according to claim 3, and at least one pharmaceutically acceptable excipient.

16. The pharmaceutical composition according to claim 15, characterized in that the composition is formulated into a liquid formulation suitable for injection or infusion, or an oral solid formulation.

17. The pharmaceutical composition according to claim 16, characterized in that the composition is formulated into a formulation which slowly releases the GIP analogue or the long-acting conjugate thereof in the composition.

18. The pharmaceutical composition according to claim 15, further comprising a GLP-1 receptor agonist, a glucagon receptor agonist, and a GLP-1/glucagon receptor coagonist.

19. A method for treating or preventing a disease selected from weight management, obesity and obesity-related diseases, all types of diabetes such as type 2 diabetes, and diabetes-related diseases, comprising administrating to a subject in need thereof, an effective amount of the long-acting conjugate of GIP analog according to claim 3.
